(19) United States
(12) Patent Application Publication
(10) Pub. No.: US 2024/0376129 A1
(43) Pub. Date: Nov. 14, 2024

(54) METALLODRUG THERAPEUTIC COMPOUNDS AND PRODRUGS OF METALLODRUG THERAPEUTIC COMPOUNDS

(71) Applicant: Dublin City University, Dublin (IE)

(72) Inventors: Andrew Kellett, Dublin (IE); Natasha McStay, Dublin (IE)

(21) Appl. No.: 18/688,719

(22) PCT Filed: Sep. 1, 2022
(86) PCT No.: PCT/EP2022/074405

§ 371 Date: Mar. 1, 2024

(30) Foreign Application Priority Data
Sep. 1, 2021 (GB) 2112479.7

(51) Int. Cl. C07F 1/00; C07D 249/04; C07D 401/14; C07D 403/14; C07D 409/14; C07D 417/14; C07D 471/04

(52) U.S. Cl. CPC C07F 1/005; C07D 249/04; C07D 401/14; C07D 403/14; C07D 409/14; C07D 417/14; C07D 471/04

(57) ABSTRACT

Disclosed herein are compounds of various formulas. In particular, the compounds are metallodrug therapeutic compounds or prodrugs of metallodrug therapeutic compounds. The metallodrug therapeutic compounds and prodrugs of metallodrug therapeutic compounds are for use in therapy, for example in the treatment and detection of cancer.

### FIELD OF THE INVENTION

The present invention relates to compounds, for example metallodrug therapeutic compounds and prodrugs of metallodrug therapeutic compounds.

### BACKGROUND OF THE INVENTION

Nucleic acids are biomolecules chiefly responsible for mediating faithful cellular replication and translation in nearly all forms of cellular life. Both DNA and RNA are therefore subjected to drug discovery efforts so that cancer, monogenetic, and pathogenic diseases can be effectively treated.

Since double helical DNA encodes genetic information, it serves as a primary target for small molecule binding agents. These interactions typically involve molecular recognition such as the curvature of the major or minor groove, the negatively charged phosphate backbone, or the space between specific base pairs (steps) required for intercalation. Binding at the molecular level broadly falls into two main categories: covalent and non-covalent. Covalent binders directly coordinate nucleic acids distorting their shape, structure and function and are often used as first line treatments against cancer; the interaction is considered permanent as it can be removed only by DNA excision repair enzymes. Non-covalent binders interact transiently but in doing so also distort shape and function as the primary structure reorganises to accommodate the ligand.

Characterising purely covalent or non-covalent interactions can be difficult as many covalent binders must firstly interact with DNA preassociativity using non-covalent interactions (kinetically fast) prior to bond formation (kinetically slow). A specific example of this effect can be traced in the interaction of the anticancer drug cisplatin (cis-[Pt(NH₃)₂Cl₂]) where non-covalent am(m)ine-phosphate interactions provide initial anchoring of the molecule prior to Pt(II) crosslinking adjacent purine bases. Conversely, several important classes of DNA damaging drugs including metallobleomycin and neocarzinostatin are potent non-covalent binding agents, but in their subsequent oxidative DNA damaging phases they activate C—H deoxyribose bonds producing double stand breaks that covalently modify the underlying nucleic acid structure.

Copper complexes have been investigated as chemotypes for treating human cancer. These investigations identified their redox properties, wide structural variability, and the bioavailability of copper. Some preclinical agents have emerged from polynuclear metal complex design. Polynuclear systems can mediate DNA damage in the absence of exogenous reductant—a property referred to as ‘self-activation’—with selective interactions such as discrimination between AT/AT and TA/TA base pairs along with discrete binding in the major groove, at helix-coil junctions, or at three-way junctions are possible through careful design. The use of polynuclear complexes therefore provides cooperative interactions at the drug-DNA interface that are not possible to achieve with simple mononuclear agents.

The present inventors reported a C₃-symmetric opioid scaffold with nucleic acid condensation properties and investigated its non-viral transfection properties (McStay, N., Molphy, Z., Coughlan, A., Cafolla, A., McKee, V., Gathergood, N. and Kellett, A. (2017) C₃-symmetric opioid scaffolds are pH-responsive DNA condensation agents. Nucleic Acids Res., 45, 527-540). The preparation of this scaffold relied on coupling three morphine, heterocodeine, oripavine or codeine molecules to a central mesitylene core. Protonation of a tertiary amine (present in the piperidine ring of each opioid) then provided an overall 3⁺ cationic charge central to DNA charge neutralisation and condensation.

C₃-symmetric tripodal scaffolds, such as those in the present invention, have been studied for their potential use as molecular sensors—specifically for the detection and recognition of inorganic phosphates which are heavily involved in DNA replication, transcription, and intracellular energy transfer (Ghosh, K., Kar, D., Joardar, S., Samadder, A., Khuda-Bukhsh, A. (2014) RSC Advances, 4, 11590-15597).

Other work has demonstrated similar C₃-symmetric scaffolds complexed to Pb(II) ions with self-assembling properties. It is thought that these self-assembling could have vast potential as drug delivery vehicles. (Najar, A. M., Tidmarsh, I. S., Ward, M. D. (2010) Cryst Eng Comm, 12, 3642-3650).

The invention of the copper(I)-catalysed click chemistry has facilitated the facile and high-yielding production of a wide range of C₃-symmetric polydentate 2-pyridyl-1,2,3-triazole ligands. The one-pot CuAAC reaction features wide substrate scope and mild reaction conditions allowing for the generation of a broad library of functional structures with extensive applications in the fields of biological and materials sciences, as demonstrated by Crowley et. al. (Crowley, J. D., Bandeem, P. H. (2009) Dalton Transactions, 39, 612-623).

Polydentate C₃-symmetric polyamine and polytriazole structures have gained traction in the realm of materials science, specifically for their use in the preparation of polyimide structures. (CN101774973B) Polyimide materials have excellent chemical properties including resistance to extreme temperatures, excellent chemical stability and exceptional tensile strength.

There is a need for new therapeutic compounds.

### SUMMARY OF THE INVENTION

According to the present invention, a C₃-symmetric approach is applied to develop therapeutically active DNA oxidants. Click chemistry is utilised to provide a modular strategy for the development of such agents.

Although click chemistry has an extensive range of applications in the field of nucleic acid click chemistry, it has not yet been widely employed to construct DNA binding metallodrugs. To facilitate the current strategy, a 1,3,5-azide mesitylene core was prepared and applied to copper(I)-catalysed azide-alkyne cycloaddition (CuAAC) click chemistry using primary, secondary and tertiary alkyne-amines to produce a library referred to as the ‘Tri-Click’ (TC) series.

TC compounds were then investigated for their ability to coordinate first row transition metals. TC1 was identified as a high-affinity copper(II) binding agent with potent DNA recognition and damaging properties.

Representative features of the present invention are set out in the following clauses, which stand alone or may be combined, in any combination, with one or more features disclosed in the text and/or drawings of the specification.

In a first aspect, the invention provides a compound of Formula (I):

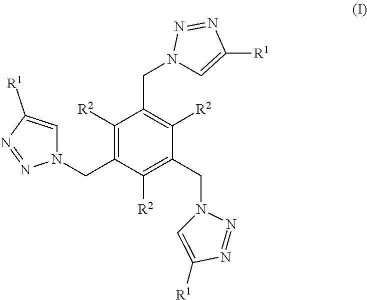

or a pharmaceutically acceptable salt thereof, wherein:
R¹ is —CH₂OH, —C(═O)OH, —C(═O)NH₂, —CH₂NH₂, —CH₂NHCH₃, —CH₂N(CH₃)₂, meta-C₆H₄NH₂, meta-C₆H₄NHCH₃, meta-C₆H₄N(CH₃)₂, para-C₆H₄N(CH₃)₂,

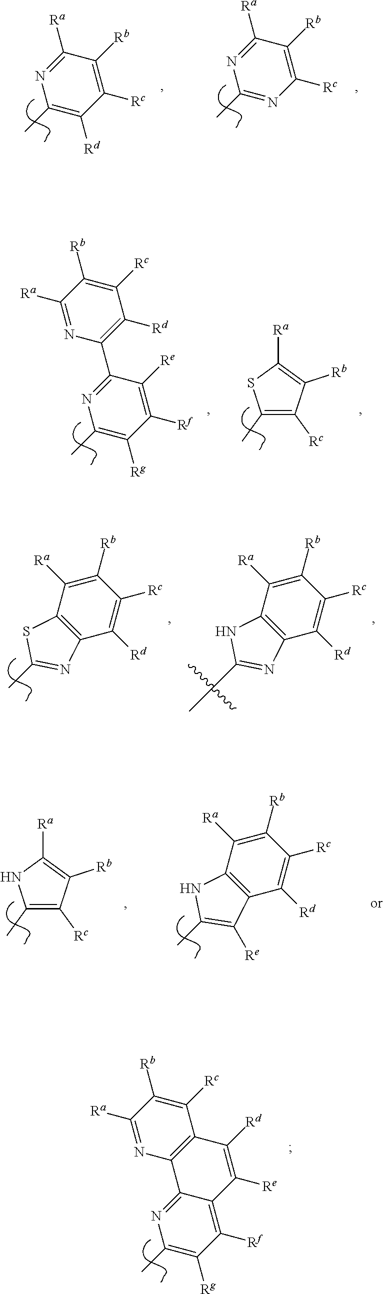

wherein:
R^{a} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{b} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{c} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{d} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{e} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{f} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃,
R^{g} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃; and,
R² at each occurrence is selected from the group consisting of: —CH₃, H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃.

In any embodiment, R² at each occurrence is —CH₃.

In any embodiment, R¹ at each occurrence is —CH₂OH, —C(═O)OH, —C(═O)NH₂, —CH₂NH₂, —CH₂NHCH₃, —CH₂N(CH₃)₂, meta-C₆H₄NH₂, meta-C₆H₄NHCH₃, meta-C₆H₄N(CH₃)₂, or para-C₆H₄N(CH₃)₂.

In any embodiment, the compound is selected from the group consisting of:

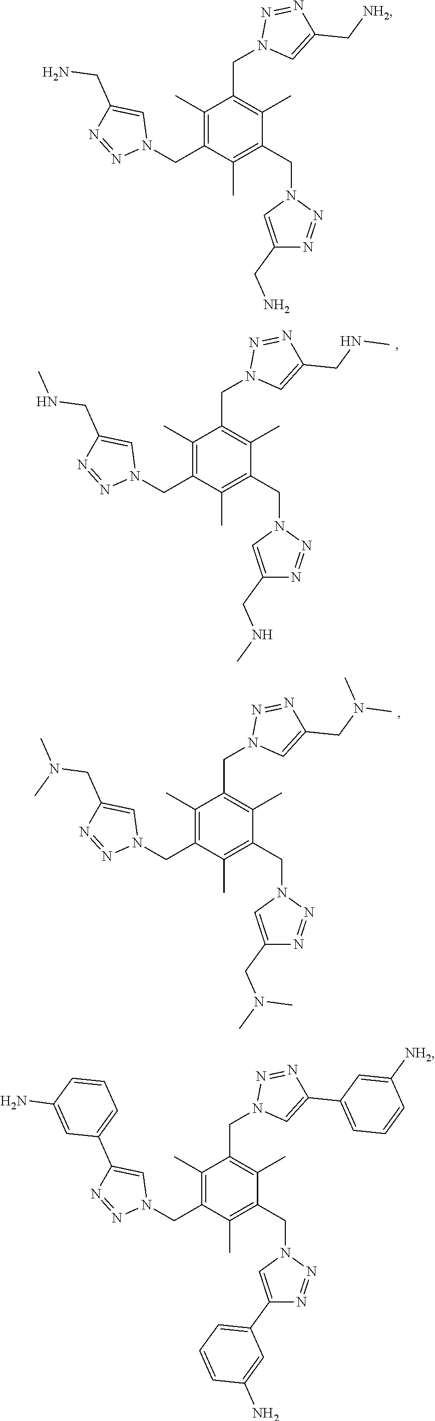

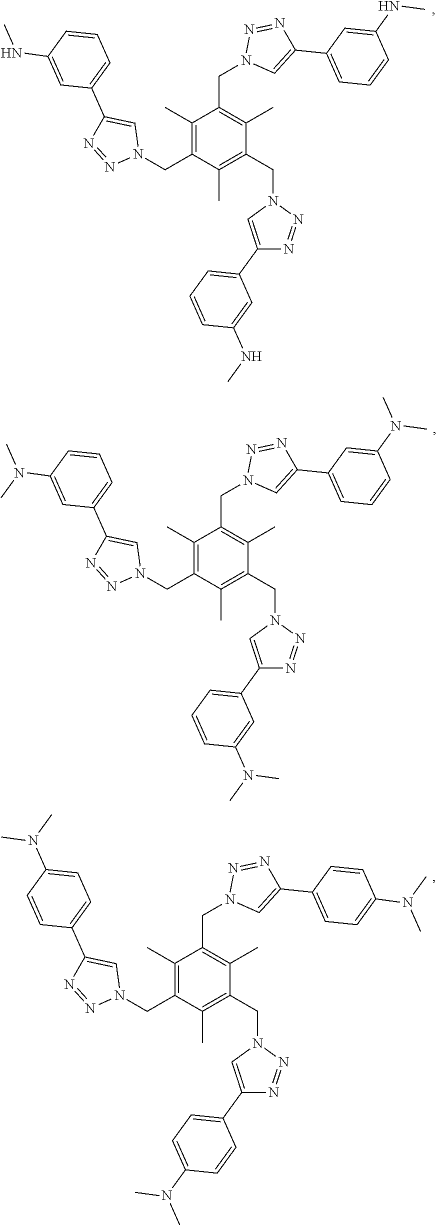

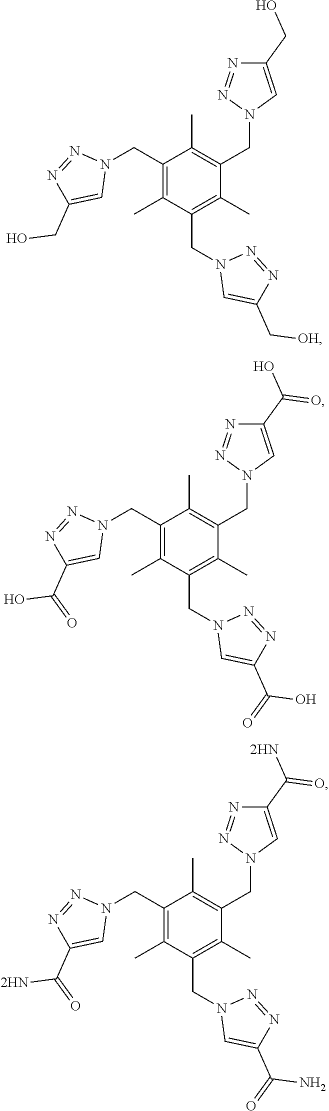

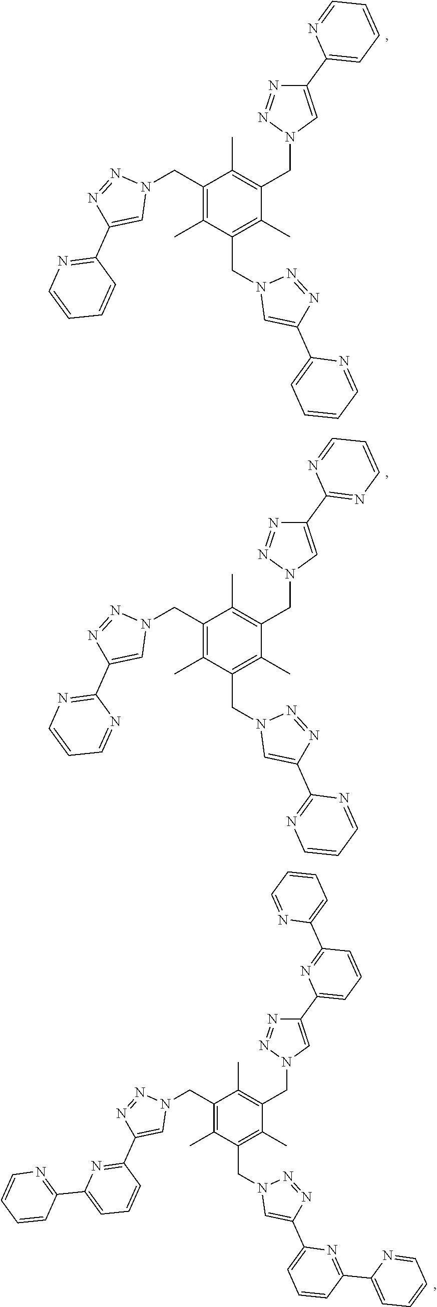

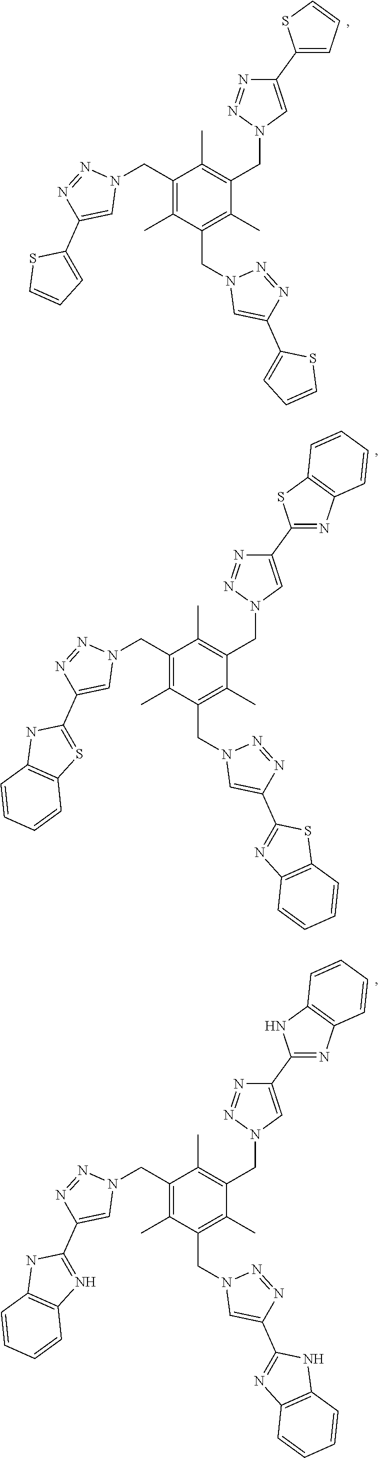

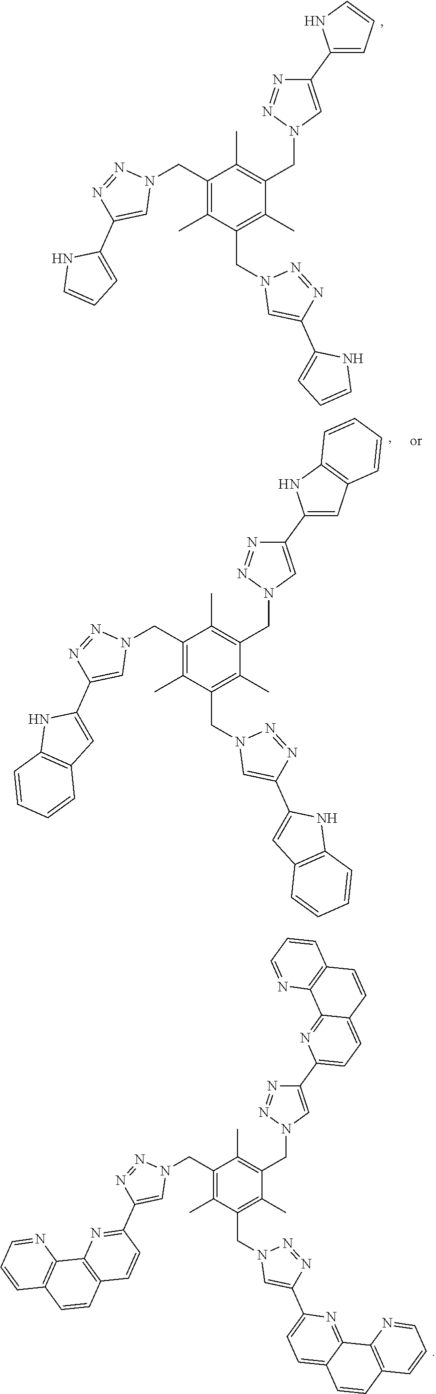

The invention also provides a compound of Formula (11):

M_{z}— Formula (I)

(II)

or a pharmaceutically acceptable salt thereof, wherein:
Formula (I) is a compound according to Formula (I) according to the invention;
M is a metal cation coordinated or chelated to the compound of Formula (I); and
z is an integer.

In any embodiment, z is selected from the group consisting of: 1, 2, 3, 4, 5 or 6.

In any embodiment, z is 3.

In any embodiment, M is: Cu²⁺, Cu¹⁺, Mn²⁺, Zn²⁺, Pt²⁺, Pt⁴⁺, Ru²⁺, Co¹⁺, Co²⁺, Fe²⁺ or Fe³⁺; wherein for each M the metal cation is any isotope of the metal cation.

In any embodiment, the compound is a compound of Formula (II-A):

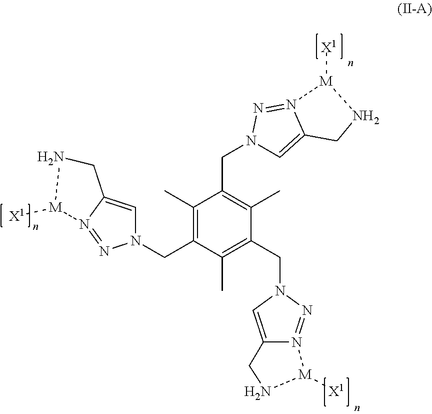

wherein:
M is Cu²⁺, Cu¹⁺, Mn²⁺, Zn²⁺, Pt²⁺, Pt⁴⁺, Ru²⁺, Co¹⁺, Co²⁺, Fe²⁺ or Fe³⁺;
X¹ is a counter ion or a water molecule or a ligand that binds to M; and,
n is an integer selected from the group consisting of: 1, 2, 3, 4, 5 or 6.

In any embodiment, the compound is a compound of Formula (II-B):

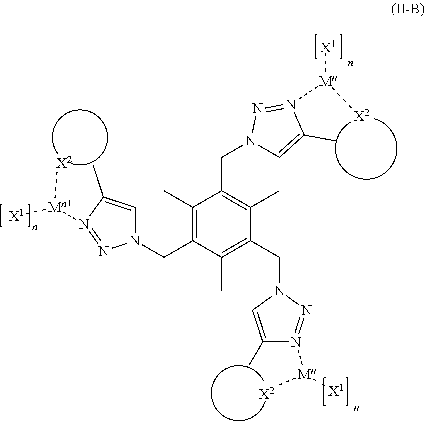

wherein:
M is Cu²⁺, Cu¹⁺, Mn²⁺, Zn²⁺, Pt²⁺, Pt⁴⁺, Ru²⁺, Co¹⁺, Co²⁺, Fe²⁺ or Fe³⁺;
X¹ is a counter ion or a water molecule or a ligand that binds to M;
n is an integer selected from the group consisting of: 1, 2, 3, 4, 5 or 6; and,
X² is a heteroatom within a cyclic substituent (optionally a cycloalkyl group including a N atom) and X² is coordinated to the metal ion M.

In any embodiment, the compound is a compound of Formula (I) of the invention that binds to one or more metal ions in an ionic solution to form a compound of Formula (II) of the invention.

The invention also provides a pharmaceutical composition comprising a compound of the invention and a pharmaceutically acceptable carrier.

The invention also provides a compound or composition of the invention for use in therapy.

The invention also provides a compound or composition of the invention for use as a medicament.

The invention also provides a compound or composition of the invention for use in the treatment or diagnosis of cancer.

The invention also provides a compound or composition of the invention for use in gene therapy.

The invention also provides a method of forming a compound of Formula (I):

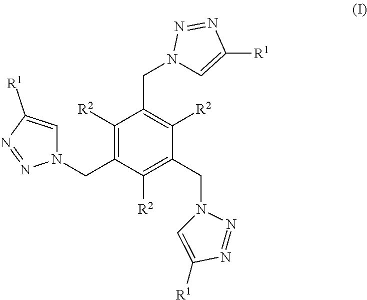

or a pharmaceutically acceptable salt thereof, wherein the method comprises the steps of:
reacting a compound of Formula (III):

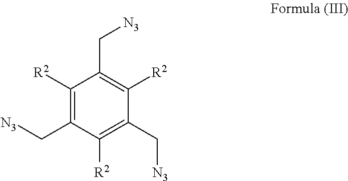

with a compound of Formula (IV)

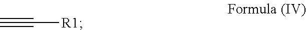

wherein:
R¹ is —CH₂OH, —C(═O)OH, —C(═O)NH₂, —CH₂NH₂, —CH₂NHCH₃, —CH₂N(CH₃)₂, meta-C₆H₄NH₂, meta-C₆H₄NHCH₃, meta-C₆H₄N(CH₃)₂, para-C₆H₄N(CH₃)₂,

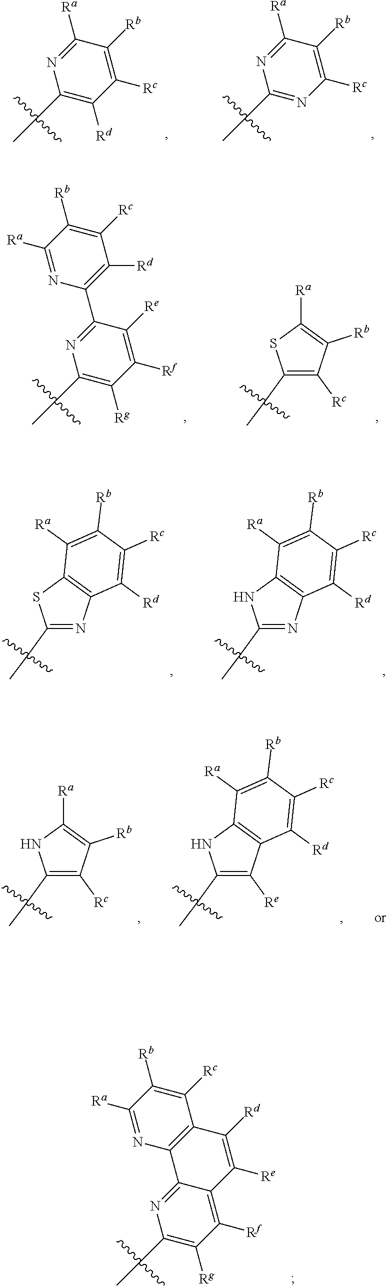

wherein:
R^{a} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{b} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{c} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{d} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{e} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{f} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃; and,
R^{g} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃; and,
R² at each occurrence is selected from the group consisting of: —CH₃, H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃.

In any embodiment, R² at each occurrence is —CH₃.

In any embodiment, the compound of Formula (IV) comprises protected substituents; optionally, wherein the compound of Formula (IV) comprises Boc protected substituents.

The invention also provides a method of forming a compound of Formula (II):

M_{z}— Formula (I)

(II)

or a pharmaceutically acceptable salt thereof, wherein:
Formula (I) is a compound as described above;
M is a metal cation coordinated or chelated to the compound of Formula (I); and
z is an integer;
   wherein the method comprises the steps of:
reacting a compound of Formula (I)

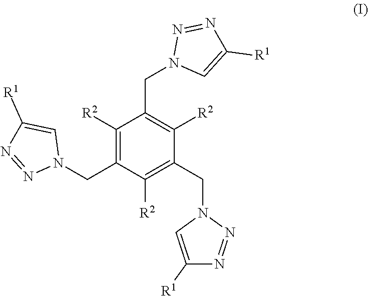

with a metal cation M.

In any embodiment, the metal cation M is: Cu²⁺, Cu¹⁺, Mn²⁺, Zn²⁺, Pt²⁺, Pt⁴⁺, Ru²⁺, Co¹⁺, Co²⁺, Fe²⁺ or Fe³⁺, wherein for each M the metal cation is any isotope of the metal cation.

### BRIEF DESCRIPTION OF THE FIGURES

Embodiments of the invention are described below with reference to the accompanying figures, in which:

FIG. 1 shows: a. General synthetic routes for the Tri-Click series. Route 1: alkyne functionalised primary and secondary amines were coupled to triazide using the CuAAC reaction. Route 2: tertiary amines where clicked with Cu(I). Route 3: Cu(I) click conditions of Boc-protected benzyl substituted amines followed by deprotection. b. Structures of successful and attempted click reactions of alkyne-amines. c. Tri-Click SAR controls. Clicked derivatives with no terminal amines TC-Br and TC-t-BuOH and simplified polyamine analogues T1-3.

FIG. 2 shows: DNA condensation of supercoiled pUC19 exposed to increasing concentrations of a, TC1 (0.5-500 μM) b, Tri-Click samples TC2-7 and c, controls at 100 and 500 μM. All reactions were carried out in neutral HEPES buffer (80 mM, pH 7.2) at 37° C. for 90 min.

FIG. 3 shows: Nuclease activity on supercoiled pUC19. DNA cleavage in the presence of 3 equivalents a, Mn(II), b, Zn(II) and c Cu(II) over 24 h. Cleavage reactions with increasing concentrations of TC1 with 3 equivalents of copper(II) nitrate over d, 1.5 h and e, 6 h. All reactions were carried out in HEPES (80 mM) with no reductant.

FIG. 4 shows: a. Competitive EtBr displacement in canonical dsDNA and alternating co-polymers. b. Apparent DNA binding constants calculated by 50% reduction in EtBr fluorescence. c. Band densitometry of Cu(II)-TC1 nuclease activity and three forms of plasmid DNA: supercoiled (form I), SSBs (form II) and DSBs (form III). d. Nuclease profiles of Cu(II)-TC1 in the presence of reductant Na-L-ascorbate (1 mM).

FIG. 5 shows: Atomic force microscopy (AFM) images of Cu(II)-TC1 treated supercoiled pUC19 (3 ng/μL) in the presence of reductant (a-d: 0.5, 1.0, 7.5, and 10 μM of Cu(II)-TC1) and absence of reductant (e-g: 5, 10 and 30 μM of Cu(II)-TC1; h: zoom of g).

FIG. 6 shows: Probing the DNA damage mechanism of Cu(II)-TC1 using ROS scavengers. DNA cleavage reactions in the presence of a, NaN₃ and tiron b, D-mannitol, DMTU and L-methionine.

FIG. 7 shows: Probing DNA damage mechanism through repair endonuclease to identify damage lesions. a, Fenton reagents, Cu(II)+H₂O₂ produce ·OH and b, Cu(II)-TC1. pUC19 DNA was preincubated with Cu(II)-TC1 or Fenton reagents for 30 min at 37° C. prior to repair enzyme addition and further incubated for 30 min. Enzymes were denatured for 20 min (50° C.) and subjected to electrophoresis.

FIG. 8 shows: DNA damaging potential and effect of antioxidants in isolated PBMCs. a. Illustration of sample collection and treatment: 1. PBMCs isolated from blood samples taken from healthy volunteers; 2. isolated cells treated with Cu(II)-TC1 in the presence or absence of ROS scavengers (pyruvate, tiron, L-histidine, D-mannitol). b. Schematic of DNA labelling process: 3. DNA with damaged bases (purple) was extracted from PBMCs; 4. BER enzymes (Fpg, Endo III or APE1, cyan) remove oxidized bases; 5. fluorescent dNTPs (aminoallyl-dUTP-ATTO-647N, red) were incorporated by DNA polymerase 1 (grey); 6. dsDNA was labelled with YOYO-1 (green). c. Representative microscopic images of labelled DNA from non-treated PBMCs (top) and Cu(II)-TC1 treated cells (bottom, TC1:Cu(II) ratio 1:1, 300:300 μM). d. DNA damage at varying TC1 concentrations with fixed Cu(II) concentration with and without radical scavengers (TC1:Cu(II) ratio 1:4, 75:300 μM). e. Identification of specific DNA damage lesions with individual BER enzymes (TC1:Cu(II) ratio 1:4, 75:300 μM).

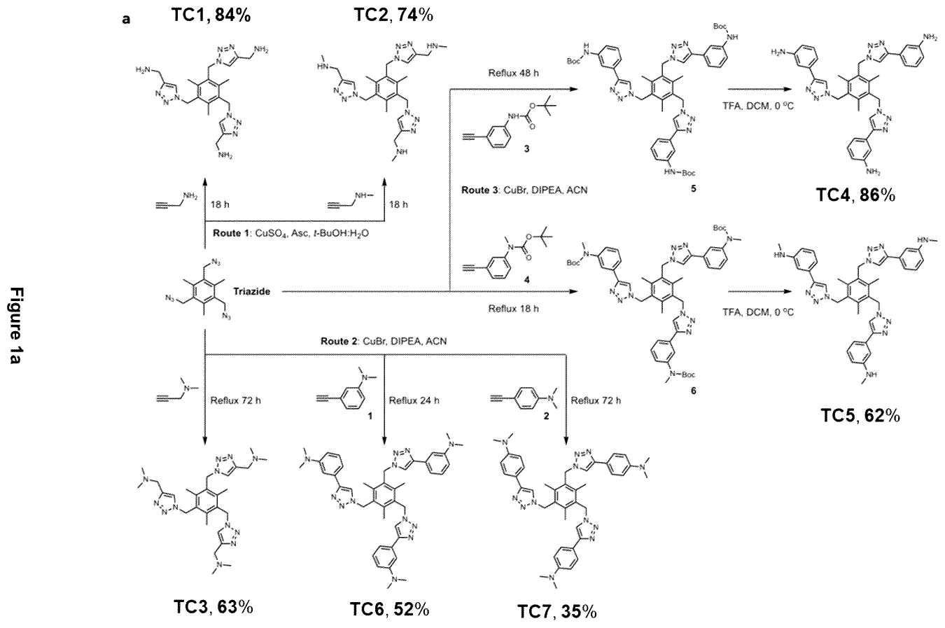

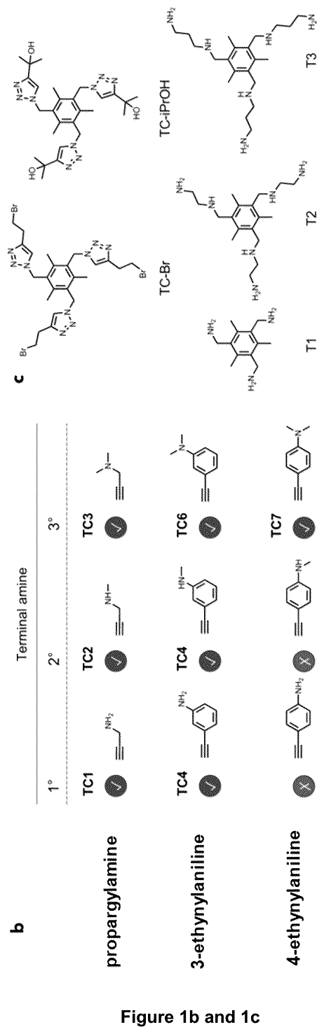

### DETAILED DESCRIPTION OF THE INVENTION

The following description and examples illustrate various embodiments of the present disclosure in detail. Those of skill in the art will recognize that there are numerous variations and modifications of this disclosure that are encompassed by its scope. Accordingly, the description of the disclosed embodiments should not be deemed to limit the scope of the present disclosure.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

As used herein, any “R” group(s) such as, without limitation, R₁, R₂, Rₐ, R_{b}, R_{c}, R_{d}, Rₑ, R_{f}, and R_{g} represent substituents that can be attached to the indicated atom. An R group may be substituted or unsubstituted. If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

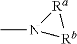

In addition, if two “R” groups are described as being “taken together” with the atom(s) to which they are attached to form a ring as an alternative, the R groups may not be limited to the variables or substituents defined previously.

As used herein, “alkyl” refers to a straight or branched hydrocarbon chain that comprises a fully saturated (no double or triple bonds) hydrocarbon group. The alkyl group may have 1 to 26 carbon atoms (whenever it appears herein, a numerical range such as “1 to 26” refers to each integer in the given range; e.g. “1 to 26 carbon atoms” means that the alkyl group may consist of 1 carbon atom, 2 carbon atoms, 3 carbon atoms, 4 carbon atoms, 5 carbon atom, 6 carbon atoms, 7 carbon atoms, 8 carbon atoms, 9 carbon atom, 10 carbon atoms, 11 carbon atoms, 12 carbon atoms, 13 carbon atoms, 14 carbon atoms, 15 carbon atoms, 16 carbon atoms, 17 carbon atoms, 18 carbon atoms, 19 carbon atoms, 20 carbon atoms, 21 carbon atoms, 22 carbon atoms, 23 carbon atoms, 24 carbon atoms, 25 carbon atoms or 26 carbon atoms, although the present definition also covers the occurrence of the term “alkyl” where no numerical range is designated). The alkyl group may also be a medium size alkyl having from 1 to 10 carbon atoms. The alkyl group could also be a lower alkyl having from 1 to 6 carbon atoms. The alkyl group of the compounds may be designated as “C₁-C₆ alkyl” or similar designations. By way of example only, “C₁-C₆ alkyl” indicates that there are one to six carbon atoms in the alkyl chain, i.e. the alkyl chain is selected from methyl, ethyl, propyl, iso-propyl, n-butyl, iso-butyl, sec-butyl, and t-butyl, pentyl (straight and branched) and hexyl (straight and branched). Typical alkyl groups include, but are in no way limited to, methyl, ethyl, propyl, isopropyl, butyl, isobutyl, tertiary butyl, pentyl (straight and branched) and hexyl (straight and branched). The alkyl group may be mono- or polysubstituted or unsubstituted. Typical substituents can be selected from —OH, —O—C₁₋₆ (optionally halo, e.g. —F, —Cl, —Br or —I)alkyl, —SH, —S—C₁₋₆ alkyl, —N₃, —NO₂, -halo (e.g. —F, —Cl, —Br or —I), —COOH, and/or —COOR_{2′} (wherein R_{2′} is substituted or unsubstituted C₁-C₂₆ alkyl).

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl. Typical substituents can be selected from —OH, —O—C₁₋₆ (optionally halo, e.g. —F, —Cl, —Br or —I)alkyl, —SH, —S—C₁₋₆ alkyl, —N₃, —NO₂, -halo (e.g. —F, —Cl, —Br or —I), —COOH, and/or —COOR_{2′} (wherein R_{2′} is substituted or unsubstituted C₁-C₂₆ alkyl).

As used herein, “aryl” refers to a carbocyclic (all carbon) mono-cyclic or multi-cyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be mono- or polysubstituted or unsubstituted. Typical substituents can be selected from —OH, —O—C₁₋₆ (optionally halo, e.g. —F, —Cl, —Br or —I)alkyl, —SH, —S—C₁₋₆ alkyl, —N₃, —NO₂, -halo (e.g. —F, —Cl, —Br or —I), —COOH, and/or —COOR_{2′} (wherein R_{2′} is substituted or unsubstituted C₁-C₂₆ alkyl).

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e. the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like. An alkenyl group may be mono- or polysubstituted or unsubstituted. Typical substituents can be selected from —OH, —O—C₁₋₆ (optionally halo, e.g. —F, —Cl, —Br or —I)alkyl, —SH, —S—C₁₋₆ alkyl, —N₃, —NO₂, -halo (e.g. —F, —Cl, —Br or —I), —COOH, and/or —COOR_{2′} (wherein R_{2′} is substituted or unsubstituted C₁-C₂₆ alkyl).

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 10 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e. the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like. An alkynyl group may be mono- or polysubstituted or unsubstituted. Typical substituents can be selected from —OH, —O—C₁₋₆ (optionally halo, e.g. —F, —Cl, —Br or —I)alkyl, —SH, —S—C₁₋₆ alkyl, —N₃, —NO₂, -halo (e.g. —F, —Cl, —Br or —I), —COOH, and/or —COOR_{2′} (wherein R_{2′} is substituted or unsubstituted C₁-C₂₆ alkyl).

As used herein, “thiol” refers to a straight or branched hydrocarbon chain containing one or more sulfur atoms, for example —SH at the end of a straight or branched hydrocarbon chain. A thiol group may be mono- or polysubstituted or unsubstituted. Typical substituents can be selected from —OH, —O—C₁₋₆ (optionally halo, e.g. —F, —Cl, —Br or —I)alkyl, —SH, —S—C₁₋₆ alkyl, —N₃, —NO₂, -halo (e.g. —F, —Cl, —Br or —I), —COOH, and/or —COOR_{2′} (wherein R_{2′} is substituted or unsubstituted C₁-C₂₆ alkyl).

As used herein, “amine” refers to a straight or branched hydrocarbon chain containing one or more nitrogen atoms, for example —NR′R″ at the end of a straight or branched hydrocarbon chain. In a primary amine, R′ and R″ are both H. In a secondary amine, R′ can be selected from the group consisting of unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl or substituted C₂-C₁₀ alkynyl, and R″ is H. In a tertiary amine, both R′ and R″ are not H and can be selected from the group consisting of unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl or substituted C₂-C₁₀ alkynyl. An amine group may be mono- or polysubstituted or unsubstituted. Typical substituents can be selected from —OH, —O—C₁-6 (optionally halo, e.g. —F, —Cl, —Br or —I)alkyl, —SH, —S—C₁₋₆ alkyl, —N₃, —NO₂, -halo (e.g. —F, —Cl, —Br or —I), —COOH, and/or —COOR₂′ (wherein R_{2′} is substituted or unsubstituted C₁-C₂₆ alkyl).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g. hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

The metal cation is generally a bioavailable metal cation. The term “bioavailable metal cation” refers to a metal cation that is readily absorbed by the body. Typically, the bioavailability of a drug is determined by comparison of the fraction of drug which is administered and the fraction of which reaches systemic circulation. Examples of bioavailable metal cations include, but are not limited to, Pt(II), Pt(IV), Cu(I), Cu(II), Ni(II), Ni(III), Hg(I), Hg(II) Ru(II), Ru(III).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e. hydrates) or common organic solvents. Unless otherwise indicated, such solvates are included in the scope of the compounds disclosed herein.

It is to be understood that where compounds disclosed herein have unfilled valencies, then the valencies are to be filled with hydrogens or isotopes thereof, e.g. hydrogen-1 (protium) and hydrogen-2 (deuterium).

It is understood that the compounds described herein can be labelled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

As used herein, the term “prodrug” generally refers to a compound, which is pharmaceutically acceptable and upon administration is converted to a desired active compound. In some embodiments, the prodrug can be therapeutically inactive until cleaved to release the active compound. The prodrug will contain an “active” component and a moiety (for example a protecting group) attached to the “active” component. Removal of some or all of the moiety will convert the prodrug from an inactive form to an active drug. This is done in the body by a chemical or biological reaction.

Depending on the moiety (for example a protecting group) attached to the “active” component, the at least one prodrug formed can be either a neutral (uncharged), a free acid, a free base or a pharmaceutically acceptable anionic or cationic salt form or salt mixtures with any ratio between positive and negative components. These anionic salt forms can include, but are not limited to, for example, acetate, I-aspartate, besylate, bicarbonate, carbonate, d-camsylate, I-camsylate, citrate, edisylate, formate, fumarate, gluconate, hydrobromide/bromide, hydrochloride/chloride, d-lactate, I-lactate, d,l-lactate, d,l-malate, I-malate, mesylate, pamoate, phosphate, succinate, sulfate, bisulfate, d-tartrate, I-tartrate, d,l-tartrate, meso-tartrate, benzoate, gluceptate, d-glucuronate, hybenzate, isethionate, malonate, methylsufate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate (mucate), galacturonate, gallate, gentisate, glutamate, glutamate, glutarate, glycerophosphate, heptanoate (enanthate), hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, or undecylenate. The cationic salt forms can include, but are not limited to, for example, sodium, potassium, calcium, magnesium, zinc, aluminum, lithium, cholinate, lysinium, ammonium, or tromethamine.

The term “pharmaceutically acceptable carriers” includes, but is not limited to, 0.01-0.1 M and preferably 0.05 M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In some embodiments, the carrier can be a) 10% PEG (polyethylene glycol) 400 (v/v)+30% (v/v) HPβCD (hydroxypropyl β-cyclodextrin), 50% w/v+60% (v/v) Sterile Water for Injection or b) 0.1% (v/v) Tween 80+0.5% (w/v) carboxymethylcellulose in water.

The term “subject” refers to a mammal, such as humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, and cats, avian species, such as chickens, turkeys, and songbirds. The subject can be, for example, a child, such as an adolescent, or an adult.

The term “treatment” refers to any treatment of a pathologic condition in a subject, such as a mammal, particularly a human, and includes: (i) preventing and/or reducing the risk of a pathologic condition from occurring in a subject which may be predisposed to the condition but has not yet been diagnosed with the condition and, accordingly, the treatment constitutes prophylactic treatment for the disease condition; (ii) inhibiting and/or reducing the speed of development of the pathologic condition, e.g., arresting its development; (iii) relieving the pathologic condition, e.g., causing regression of the pathologic condition; or (iv) relieving the conditions mediated by the pathologic condition and/or symptoms of the pathologic condition. Treatment of subjects who have previously and/or are currently, and/or are about to receive a cancer therapy are contemplated herein.

The term “therapeutically effective amount” refers to that amount of a compound of the invention that is sufficient to effect treatment, when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

### EXAMPLES

### General Remarks

Chemicals and reagents were sourced from Sigma-Aldrich and Tokyo Chemical Industry (TCI) and were used without any further purification. HPLC grade chloroform, methanol, and acetonitrile were used without further purification. All other solvents were used as supplied. All novel compounds were characterised by melting point (MP) (when appropriate), nuclear magnetic resonance (NMR) spectroscopy, attenuated total reflectance (ATR) Fourier transform infrared (FTIR) spectroscopy, and electron spray ionisation mass spectrometry (ESI-MS). Thin layer chromatography was performed on Fluka Silica gel (60 F254) coated on aluminium plates and visualised using UV light. Davisil 60 Å silica gel was used for column chromatography. ¹H and ¹³C NMR spectra were obtained on a Bruker AC 400 MHz and 600 MHz NMR spectrometer. FT-IR spectra were collected on Perkin Elmer Spectrum Two spectrometer. Electrospray ionisation mass spectra were recorded using a Thermo Fisher Exactive Orbitrap mass spectrometer coupled to an Advion TriVersa Nanomate injection system with samples prepared in 100% HPLC-grade acetonitrile or methanol.

Caution! Sodium azide is acutely toxic and is an explosion hazard. Refer to organic azide stability prior to the preparation of any azido compounds. The total number of nitrogen atoms in a final organic azide should not exceed that of carbon. Organic azides with C/N ratio of <1 should never be isolated. It may be synthesized if the azide is a transient intermediate species and the limiting reagent in the reaction mixture and is limited to a maximum quantity of 1 gram. Each azido compound should be individually evaluated.

GenElute-Mammalian Genomic DNA Miniprep Kits, tiron, D-mannitol, sodium pyruvate, APTES, ATMS, β-mercaptoethanol, L-histidine, calf-thymus (ctDNA, Ultra-Pure 15633019) was purchased from Invitrogen while, poly[d(A-T)₂)](P0883) and poly[d(G-C)₂] (P9389) were purchased from Sigma-Aldrich. CutSmart buffers, pUC19 plasmid (N3041) and repair enzymes—Fpg (M0240S), Endo III (M0268S), Endo IV (M0304S), Endo V (M0305S), hAAG (M0313S) and APE1 (M0282S)—were purchased from New England Biolabs. Aminoallyl-dUTP-ATTO-647N and YOYO-1 were purchased from Jena Bioscience and Invitrogen, respectively.

The pH was monitored by a Mettler Toledo InLab Expert Pro-ISM pH probe. DNA binding analysis was conducted on a Bio-Tek, Synergy HT fluorescent microplate reader with Gen5 software. UV-visible spectrometry studies were carried out on a Shimadzu UV-2600. AFM images where captured on Bruker Dimension Icon AFM equipped with super sharp silicon cantilevers (SSS—NCHR, Windsor Scientific Ltd). Zeiss Observer. Z1 equipped with an Andor iXON Ultra EMCCD camera was used to obtain the fluorescence images.

### Ligand Preparation

2,4,6-Tris-(azidomethyl)-mesitylene (triazide). To a solution of 2,4,6-tris-(bromomethyl)-mesitylene (1.025 g, 2.56 mmol) in DMF (25 mL), sodium azide (1.00 g, 15.38 mmol) was added in portions over ice over a period of 20 min. (Caution! Sodium azide is acutely toxic and is an explosion hazard. Refer to organic azide stability prior to the preparation of any azido compounds). The reaction was stirred on ice for 1 h prior to stirring at rt for 23 h. The reaction was quenched with 8 mL of H₂O, and extracted with EtOAc (3×20 mL). The combined organic phase was washed with H₂O (5×5 mL) and the organic layer dried over MgSO₄, filtered and solvents removed by rotary evaporation. The sample was recrystallized from a solvent system of hex:EtOAc (5:1) to afford the triazide (0.677 g, 2.37 mmol, 93%) as a white crystalline solid. ¹H NMR (600 MHz, CDCl₃) δ: 4.50 (s, 6H), 2.46 (s, 9H). ¹³C NMR (151 MHz, CDCl₃) δ: 138.15, 130.88, 77.27, 77.06, 76.85, 48.94, 16.50. ¹H and ¹³C NMR were in agreement with literature data. IR (ATR, cm⁻¹): 2901, 2085, 1678, 1572, 1449, 1232, 1072, 859, 698, 640, 552.

3-Ethynyl-N,N-dimethylaniline (1). To a solution of 3-ethynylaniline (1.034 g, 8.83 mmol) and caesium carbonate (8.306 g, 25.5 mmol) in dry DMF (50 mL) under nitrogen, methyl iodide was added drop wise over 15 min (1.7 mL, 26.0 mmol). The reaction was heated to 40° C. for 24 h and was monitored by TLC (Hex:EtOAc). From the complete conversion by TLC, the reaction was cooled and diluted with H₂O (100 mL) and extracted with EtOAc (3×30 mL). The organic layers were combined and washed with H₂O (3×10 mL) and finally with brine solution. The organic layer was dried over MgSO₄ and reduced to dryness. Product was purified by column chromatography (SiO₂, Hex:EtOAc, 9:1) as a yellow liquid (383 mg, 2.64 mmol, 30%). ¹H NMR (400 MHz, CDCl₃) δ: 7.22-7.16 (m, 1H), 6.90-6.84 (m, 2H), 6.74 (ddd, J=8.4, 2.6, 1.0 Hz, 1H), 3.03 (s, 1H), 2.95 (s, 6H). The ¹H NMR spectrum agreed with literature data.

4-Ethynyl-N,N-dimethylaniline (2). To a solution of 4-ethynylaniline (1.003 g, 8.56 mmol) and cesium carbonate (8.345 g, 25.6 mmol) in dry DMF (50 mL) under nitrogen, methyl iodide was added drop wise over 15 min (1.6 mL, 26.0 mmol). The reaction was heated to 40° C. for 24 h and monitored by TLC (Hex:EtOAc). The reaction was cooled and diluted with H₂O (100 mL) and extracted with EtOAc (3×30 mL). The organic layers were combined and washed with H₂O (3×10 mL) and finally with brine solution. The organic layer was dried over MgSO₄ and reduced to dryness. Product was purified by column chromatography (SiO₂, Hex:EtOAc, 9:1) as a colourless liquid (383 mg, 2.64 mmol, 31%). ¹H NMR (400 MHz, CDCl₃) δ: 7.39 (dt, 2H), 6.65 (dt, 2H), 3.00 (s, 7H). The ¹H NMR spectrum was in agreement with literature data.

N-Boc-3-ethynylaniline (3). To a solution of 3-ethynylaniline (5.040 g, 43.02 mmol) in dry THF (80 mL), di-tert-butyl-dicarbonate (10.112 g, 46.33 mmol) in THF (10 mL) was added dropwise. Reaction was refluxed for 18 h and monitored by TLC. Solvent was reduced under pressure and the resulting material was purified by column chromatography (Hex:EtOAc) to yield the product as a colourless liquid (8.786 g, 40.44 mmol, 94% yield). ¹H NMR (600 MHz, CDCl₃) δ: 7.37 (d, J=7.3 Hz, 1H), 7.24 (t, J=7.8 Hz, 1H), 7.17 (dt, J=7.6, 1.3 Hz, 1H), 6.58 (s, 1H), 3.06 (s, 1H), 1.53 (s, 9H). ¹H NMR spectrum was in agreement with literature data.

Boc-3-ethynyl-N-methylaniline (4). Sodium hydride (3 eq, 14.8 mmol) was prepared in the reaction vessel under nitrogen flux and washed with dry hexane, suspended in dry DMF (40 mL) over ice. A solution of boc-3-ethynylaniline (1.038 g, 4.93 mmol) in DMF (10 mL) was added slowly over 30 min to the reaction under a nitrogen atmosphere and the resulting solution was allowed to stir at rt for 2 h. Methyl Iodide (1.54 mL, 5 eq, 24.7 mmol) was added to the reaction slowly over 5 min and reaction left stirring for 4 h. The reaction was quenched slowly with H₂O at 0° C. The solution was diluted with H₂O (50 mL) and extracted with EtOAc (4×30 mL). The organic layers were combined and further washed with H₂O (5×10 mL) and finally with brine. The organic layer was dried over MgSO₄, filtered and solvents removed by rotary evaporation. The crude product was purified by column chromatography (SiO₂, 9.5:0.5 Hex:EtOAc) as an off white solid (850 mg, 3.68 mmol, 75%). ¹H NMR (400 MHz, CDCl₃) δ: 7.38 (q, J=1.4 Hz, 1H), 7.31-7.26 (m, 3H), 3.27 (s, 3H), 3.09 (s, 1H), 1.47 (s, 9H). The ¹H NMR spectrum was in agreement with literature data.

### Boc-Click Reactions

Tri-Click boc-3-ethynylaniline (5). To a solution of triazide (0.285, 1.00 mmol) and DIPEA (0.174 mL, 1.00 mmol) in degassed ACN (20 mL), CuBr (0.144 g, 1.00 mmol) was added slowly under nitrogen atmosphere. The solution was stirred for 15 min and boc-3-ethynylaniline (3, 0.679 g, 3.13 mmol) was added dropwise as a solution in ACN (5 mL) to the reaction. The reaction was refluxed for 50° C. for 48 h, until the complete conversion was observed by TLC. The reaction was allowed to cool and ACN removed by reduced pressure. The resulting crude material was suspended in 0.1 M EDTA solution (pH 8) and heated to reflux for 1 h. The solution was allowed to cool and extracted with DCM (3×50 mL). The organic layers were combined and washed with H₂O (3×30 mL) and brine. The organic solution was dried over MgSO₄ and reduced to dryness. The crude product was column purified (SiO₄ DCM:MeOH, 9:1) resulting in the title product as cream solid (505 mg, 0.538 mmol, 54%). ¹H NMR (400 MHz, CDCl₃) δ: 7.77 (s, 3H), 7.57 (s, 3H), 7.51 (dd, J=7.6, 1.4 Hz, 3H), 7.41-7.37 (m, 3H), 7.31 (t, J=7.9 Hz, 3H), 6.80 (s, 3H), 5.74 (s, 6H), 2.49 (s, 9H), 1.52 (s, 27H). Amine deprotection detailed below.

Tri-Click boc-3-ethynyl-N-methylaniline (6). To a solution of triazide (0.286, 1.00 mmol) and DIPEA (0.174 mL, 1.00 mmol) in degassed ACN (20 mL), CuBr (0.144 g, 1.00 mmol) was added slowly under nitrogen atmosphere. The solution was stirred for 15 minutes and boc-3-ethynyl-N-methylaniline (4, 0.718 g, 3.11 mmol) was added dropwise as a solution in ACN (5 ml) to the reaction. The reaction was refluxed for 18 h, until the complete conversion was observed by TLC. The reaction was allowed to cool and ACN removed by reduced pressure. The resulting crude was suspended in 0.1 M EDTA solution (pH 8) and heated to reflux for 1 h. The solution was allowed to cool and extracted with DCM (3×50 mL). The organic layers were combined and washed with H₂O (3×30 mL) and brine. The organic solution was dried over MgSO₄ and reduced to dryness. The crude product was column purified (SiO₄ DCM:MeOH, 9:1) and resulted in a white solid (866 mg, 0.88 mmol, 88%). ¹H NMR (400 MHz, CDCl₃) δ: 7.74 (t, J=1.9 Hz, 3H), 7.55 (d, J=10.4 Hz, 6H), 7.35 (t, J=7.8 Hz, 3H), 7.22 (ddd, J=8.0, 2.3, 1.1 Hz, 3H), 5.75 (s, 6H), 3.29 (s, 9H), 2.54 (s, 9H), 1.47 (d, J=2.8 Hz, 27H). Amine deprotection procedure detailed below.

### Tri-Click Reactions

Tri-Click propargylamine (TC1). To a solution of triazide (0.214 g, 0.75 mmol) in degassed t-BuOH:H₂O (1:1, 6 mL), propargylamine (147 μL, 2.29 mmol) and Na-L-ascorbate (5%, 0.15 mmol) was added and stirred under a nitrogen atmosphere for 15 min, prior to the addition of a solution of CuSO₄ (1%, 0.03 mmol) in t-BuOH:H₂O (1:1, 2 mL) which was added dropwise over 10 min. The reaction was stirred at 25° C. for 18 h. The solvent was reduced to 3 mL under a stream of nitrogen and cooled on ice. The resulting precipitate was collected by vacuum filtration and washed with ice cold EDTA (0.1 M, 3×8 mL) and ice-cold diethyl ether (3×10 mL). A yellow solid was recovered in good yield, (284 mg, 0.63 mmol, 84%). MP: 119-120° C. ¹H NMR (600 MHz, D₂O) δ: 7.66 (s, 3H), 5.64 (s, 6H), 3.77 (s, 6H), 2.21 (s, 9H). ¹³C NMR (151 MHz, D₂O) δ: 148.32, 140.12, 130.00, 122.69, 49.03, 35.46, 15.51. IR (ATR, cm⁻¹): 3124, 2982, 1666, 1603, 1442, 1381, 1328, 1215, 1120, 1046, 949, 802. ESI-MS m/z: [M+H]⁺ Cald for C₂₁H₃₁N₁₂⁺: 451.56; found 451.6.

Tri-Click N-methyl-propargylamine (TC2). To a solution of triazide (0.430 g, 1.5 mmol) in degassed t-BuOH:H₂O (1:1, 8 mL), N-methyl-propargylamine (380 μL, 4.52 mmol) and Na-L-ascorbate (10%, 0.45 mmol) was added and stirred under a nitrogen atmosphere for 15 min. A solution of CuSO₄ (1%, 0.045 mmol) in t-BuOH:H₂O (1:1, 2 mL) was added dropwise over 10 min and stirred at 25° C. for 18 h. The solvent was removed under reduced pressure and the product was suspended in 0.1 M EDTA solution (pH 8) and extracted with DCM (3×50 mL). The organic layers were combined and washed with ice cold H₂O (3×20 mL) and brine (3×30 mL). The organic solution was dried over MgSO₄ and solvent removed under reduced pressure. A white solid was recovered (0.548 g, 1.13 mmol, 74%). MP: 197-199° C. ¹H NMR (600 MHz, CDCl₃) δ: 7.24 (s, 3H), 5.63 (s, 6H), 3.82 (s, 6H), 2.44 (s, 9H), 2.38 (s, 9H). ¹³C NMR (151 MHz, CDCl₃) δ: 146.83, 139.73, 130.66, 120.91, 48.88, 46.84, 36.16, 16.57. IR (ATR, cm⁻¹): 3066, 2981, 1634, 1550, 1447, 1380, 1331, 1261, 1207, 1131, 1047, 811. ESI-MS m/z: [M+H]⁺ Cald for C₂₄H₃₆N₁₂⁺: 493.33; found 493.2.

Tri-Click N,N-dimethyl-propargylamine (TC3). To a solution of triazide (0.286 1.00 mmol) and DIPEA (0.174 mL, 1.00 mmol) in degassed ACN (20 mL), CuBr (0.144 g, 1.00 mmol) was added slowly under nitrogen atmosphere. The solution was stirred for 15 minutes and N,N-dimethyl-propargylamine (0.340 mL, 3.16 mmol) was added dropwise as a solution in ACN (5 mL) to the reaction. The reaction was refluxed for 72 h, until the complete conversion was observed by TLC. The reaction was allowed to cool and ACN removed by reduced pressure. The resulting mixture was suspended in 0.1 M EDTA solution (pH 8) and heated to reflux for 1 h. The solution was allowed to cool and extracted with DCM (3×50 mL). The organic layers were combined and washed with H₂O (3×20 mL) and brine (3×30 mL). The organic solution was dried over MgSO₄ and solvent removed under reduced pressure. The resulting product was column purified (SiO₄ DCM:MeOH, 9:1) and isolated as an orange solid (338 mg, 0.63 mmol, 63%). MP: 227-229° C. ¹H NMR (600 MHz, (CD₃)₂SO) δ: 7.75 (s, 3H), 5.67 (s, 6H), 3.43 (s, 6H), 2.39 (s, 9H), 2.09 (s, 18H). ¹³C NMR (151 MHz, (CD₃)₂SO) δ: 144.10, 139.56, 131.42, 123.66, 54.02, 48.75, 45.04, 16.71. Anal. Cal. for C₂₇H₄₂N₁₂: C, 60.65; H, 7.92; N, 31.43. % Found: C, 59.34, H, 7.72, N, 30.45. IR (ATR, cm⁻¹): 3111, 3070, 2977, 2939, 2814, 2762, 1455, 1380, 1336, 1297, 1255, 1213, 1174, 1135, 1037, 1016, 840, 812, 798, 704. ESI-MS m/z: [M]⁺ Cald for C₂₇H₄₂N₁₂: 534.7; found 535.3.

Tri-Click 3-ethynylaniline (TC4). To a solution of Tri-Click boc-3-ethynylaniline (5) (0.505 mg, 0.539 mmol) in DCM (10 mL) TFA (0.615 μL, 8 mmol) was added slowly over ice. The solution was allowed to stir over ice and monitored by TLC to ensure complete removal of Boc group. The solvent and excess TFA were removed under a stream of nitrogen, residue was dissolved in HCl (aq) (1.0 M, 10 mL) and washed with DCM (3×20 mL). DCM (20 mL) and water (20 mL) was added to the aqueous fraction and the pH was adjusted to 8 with NaOH (1 M) while stirring. The organic fraction was collected, and the aqueous layer was extracted with DCM (2×10 mL). The combined organic fractions were dried (MgSO₄), filtered and the solvent was removed under reduced pressure. The resulting product was column purified (SiO₄ DCM:MeOH, 9:1) to afford a white solid (0.295 g, 0.464 mmol, 86%). MP: 228-230° C. ¹H NMR (600 MHz, (CD₃)₂SO) δ: 8.24 (s, 3H), 7.12 (t, J=2.0 Hz, 3H), 7.06 (t, J=7.8 Hz, 3H), 6.97 (dt, J=7.5, 1.2 Hz, 3H), 6.54 (ddd, J=8.0, 2.4, 1.1 Hz, 3H), 5.74 (s, 6H), 3.35 (s br, 6H), 2.48 (s, 9H). ¹³C NMR (151 MHz, (CD₃)₂SO) δ: 157.87, 157.66, 148.37, 146.77, 139.40, 131.11, 130.81, 129.27, 120.62, 113.88, 113.57, 110.82, 48.59, 16.42 IR (ATR, cm⁻¹): 3347, 1674, 1611, 1589, 1430, 1182, 1123, 1045, 836, 802, 782, 722, 691. ESI-MS m/z: [M+H]⁺ Cald for C₃₆H₃₇N₁₂⁺: 637.3; found 637.3.

Tri-Click 3-ethynyl-N-methylaniline (TC5). The removal of the protecting group was carried as stated above. Briefly, to a solution of Tri-Click boc-3-ethynyl-N-methylaniline (6) (0.850 g, 0.87 mmol) in DCM (15 mL) TFA (1 mL, 13.05 mmol) was added slowly over ice. The solution was allowed to stir over ice and monitored by TLC to ensure complete removal of Boc group. No alterations were made to the workup as detailed previously. The resulting product was column purified (SiO₄ DCM:MeOH, 9:1). Solvent was removed under reduced pressure and foamed with THF to afford an extremely hygroscopic white solid (0.366 g, 0.539 mmol, 62%). Product is stored under argon. ¹H NMR (600 MHz, (CD₃)₂SO) δ: 8.31 (s, 3H), 7.17-7.13 (m, 6H), 7.08 (dt, J=7.6, 1.3 Hz, 3H), 6.59 (ddd, J=8.0, 2.4, 1.0 Hz, 3H), 5.75 (s, 6H), 2.73 (s, 9H), 2.49 (s, 9H). ¹³C NMR (151 MHz, (CD₃)₂SO) δ: 146.81, 139.50, 131.32, 130.87, 129.40, 120.87, 119.79, 117.83, 115.86, 114.22, 113.89, 112.54, 109.16, 48.63, 30.42, 16.50, 1.17. IR (ATR, cm⁻¹): 3112, 1670, 1435, 1177, 1123, 837, 7800, 722, 710, 599, 518. ESI-MS m/z: [M+H]⁺ Cald for C₃₉H₄₃N₁₂⁺: 679.4; found 679.4. Note: hygroscopic, requires storage under argon

Tri-Click 3-ethynyl-N,N-dimethylaniline (TC6). To a solution of triazide (0.286, 1.00 mmol) and DIPEA (0.174 mL, 1.00 mmol) in degassed ACN (20 mL), CuBr (0.144 g, 1.00 mmol) was added slowly under nitrogen atmosphere. The solution was stirred for 15 min and 3-ethynyl-N,N-dimethylaniline (1, 0.450 g, 3.09 mmol) was added dropwise as a solution in ACN (5 mL) to the reaction. The reaction was heated to 50° C. under nitrogen for 24 h, until complete conversion was observed by TLC. The reaction was allowed to cool and ACN removed by reduced pressure. The resulting crude was suspended in 0.1 M EDTA solution (pH 8) and heated to reflux for 1 h. The solution was allowed to cool and extracted with DCM (3×50 mL). The organic layers were combined and washed with H₂O (3×30 mL) and brine. The organic solution was dried over MgSO₄ and reduced to dryness. The crude product was column purified (SiO₄ DCM:MeOH, 9:1) and isolated as beige powder (399 mg, 0.52 mmol, 52%). MP: 259-260° C. ¹H NMR (600 MHz, CDCl₃) δ: 7.46 (s, 3H), 7.29 (dd, J=2.7, 1.5 Hz, 3H), 7.21 (t, J=8.2 Hz, 3H), 6.95 (ddd, J=7.5, 1.5, 0.9 Hz, 3H), 6.69 (ddd, J=8.4, 2.7, 0.9 Hz, 3H), 5.71 (s, 6H), 2.98 (s, 18H), 2.51 (s, 9H). ¹³C NMR (151 MHz, CDCl₃) δ: 151.13, 148.74, 139.96, 131.00, 130.98, 129.58, 118.98, 114.19, 112.69, 109.75, 49.21, 40.78, 16.96. IR (ATR, cm⁻¹): 1605, 1582, 1497, 1442, 1350, 1223, 1182, 1042, 986, 857, 780, 692, 461. Anal. Cal. for C₄₂H₄₈N₁₂: C, 69.97; H, 6.71; N, 23.31. % Found: C, 69.30; H, 6.66; N, 22.70. ESI-MS m/z: [M+H]⁺ Cald for C₄₂H₄₉N₁₂⁺: 721.4; found 721.4.

Tri-Click 4-ethynyl-N,N-dimethylaniline (TC7). To a solution of triazide (0.286, 1.00 mmol) and DIPEA (0.174 mL, 1.00 mmol) in degassed ACN (20 mL), CuBr (0.144 g, 1.00 mmol) was added slowly under nitrogen atmosphere. The solution was stirred for 15 min and 4-ethynyl-N,N-dimethylaniline (2, 0.454 g, 3.13 mmol) was added dropwise as a solution in ACN (5 mL). The reaction was refluxed for 72 h, until the complete conversion was observed by TLC. The reaction was allowed to cool and ACN removed by reduced pressure. The resulting crude was suspended in 0.1 M EDTA solution (pH 8) and heated to reflux for 1 h. The solution was allowed to cool and extracted with DCM (3×50 mL). The organic layers were combined and washed with H₂O (3×30 mL) and brine. The organic solution was dried over MgSO₄ and reduced to dryness. The crude product was column purified (SiO₄ DCM:MeOH, 9:1) and isolated as a cream solid (249 mg, 0.345 mmol, 35%). MP: 284-286° C. ¹H NMR (600 MHz, (CD₃)₂SO) δ: 8.09 (s, 3H), 7.63 (d, J=8.8 Hz, 6H), 6.71 (d, J=8.9 Hz, 6H), 5.73 (s, 6H), 2.91 (s, 18H), 2.47 (s, 9H). ¹³C NMR (151 MHz, (CD₃)₂SO) δ: 150.41, 147.27, 139.75, 131.35, 126.58, 119.39, 119.02, 112.66, 49.00, 16.84. ATR-IR (cm⁻¹): 2851, 1619, 1559, 1443, 1348, 1220, 1170, 1040, 947, 812, 695, 533. ESI-MS m/z: [M+H]⁺ Cald for C₄₂H₄₉N₁₂⁺: 721.4; found 721.4.

The compounds referred to as TC1, TC2, TC3, TC4, TC5, TC6 and TC7 are summarised in Table 1 below:

TABLE 1
TC series structures

| Compound name | Structure |
|---|---|
| TC1 | 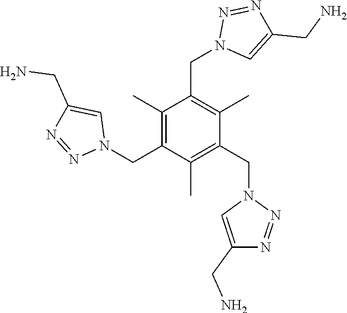 [SMILES: Cc1c(Cn2cc(CN)nn2)c(C)c(Cn2cc(CN)nn2)c(C)c1Cn1cc(CN)nn1] |
| TC2 | 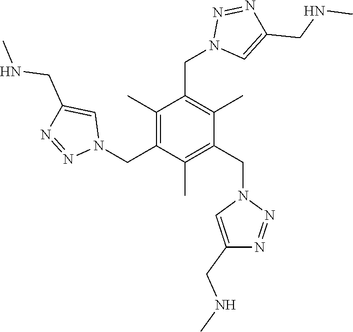 |
| TC3 | 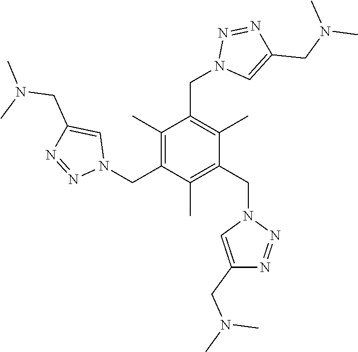 |
| TC4 | 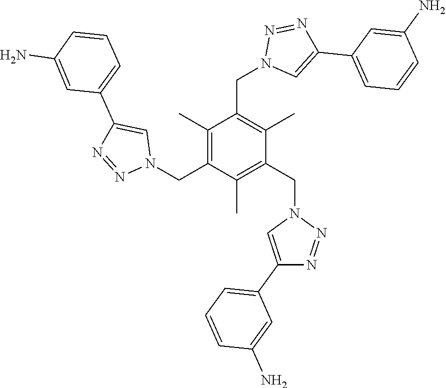 [SMILES: Cc1c(Cn2cc(-c3cccc(N)c3)nn2)c(C)c(Cn2cc(-c3cccc(N)c3)nn2)c(C)c1Cn1cc(-c2cccc(N)c2)nn1] |
| TC5 | 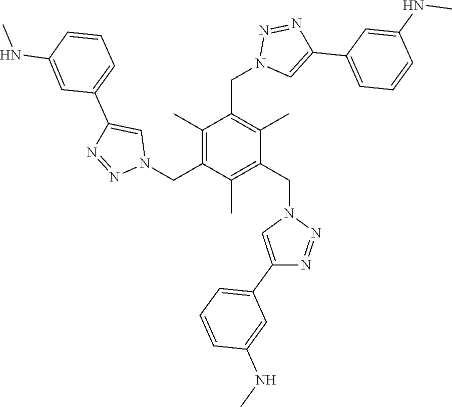 |
| TC6 | 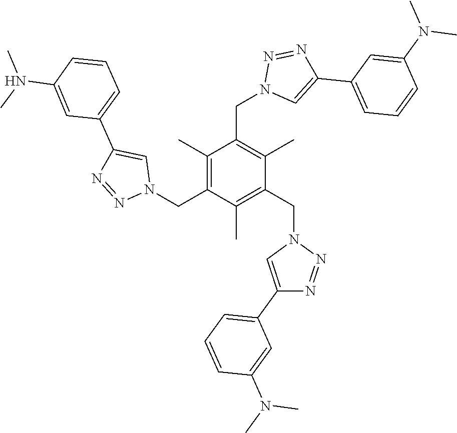 |
| TC7 | 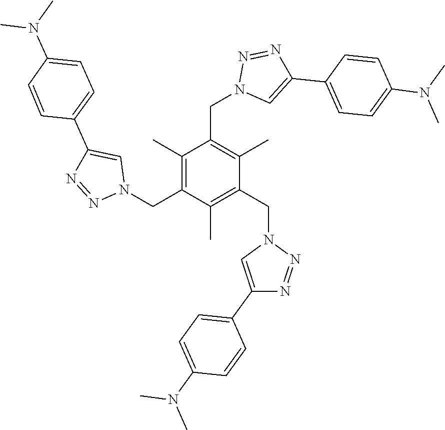 |

### Investigation of the Para-Substituted 4-Ethynylanilines

N-Boc-4-ethynylaniline (7). To a solution of 4-ethynylaniline (5 mmol) in dry THF (20 mL), di-tert-butyl dicarbonate (15 mmol) was added dropwise. Reaction was refluxed under nitrogen for 18 h. Monitored by TLC (Hex:EtOAc 9:1) until the complete conversion of product was observed. The reaction was allowed to cool to rt and solvent removed under reduced pressure. Crude yellow oil was subjected to column chromatography (SiO₂, Hex:EtOAc, 9.5:0.5-9:1) to yield the pure product as a colourless liquid in high yield (0.98 g, 4.51 mmol, 90% yield). ¹H NMR (400 MHz, CDCl₃) δ: 7.47-7.31 (m, 4H), 6.64 (s, 1H), 3.04 (s, 1H), 1.53 (s, 9H). The H NMR spectra was in agreement with literature data.

### Tri-Click Non-Amine Controls

Tri-Click 4-bromo-1-butyne (TC-Br; comparative example). To a solution of triazide (0.100 g, 0.35 mmol) in 10 mL THF:H₂O (1:1), Na-L-ascorbate (5%) was added followed by CuSO₄ (1%) under a nitrogen atmosphere. To this solution 4-bromo-1-butyne (0.1 mL, 1.05 mmol) was added. The reaction was allowed to stir at rt for 24 h, a white precipitate was observed after 8 h. On the completion of the reaction, the white precipitate was collected by vacuum filtration and washed with diethyl ether to afford the title compound as a white solid (0.239 g, 0.35 mmol, 85%). MP: decomposed at 269° C. ¹H NMR (400 MHz, CDCl₃) δ: 7.10 (s, 3H), 5.58 (s, 6H), 3.56 (t, J=6.7 Hz, 6H), 3.17 (t, J=6.7 Hz, 6H), 2.35 (s, 9H). ¹³C NMR (151 MHz, CDCl₃) δ: 144.91, 139.70, 130.71, 121.10, 77.24, 77.03, 76.82, 48.93, 31.81, 29.31, 29.28, 16.67, 16.63. Anal. Cal. for C₂₄H₃₀Br₃N₉: C, 42.13; H, 4.42; N, 18.42; Br, 35.03. % Found: C, 42.41; H, 4.38; N, 18.50; Br, 34.65. IR (ATR, cm⁻¹): 3065, 1558, 1441, 1259, 1216, 1147, 1046, 925, 880, 812, 667, 553. ESI-MS m/z: [M+H]⁺ Cald for C₂₄H₃₁Br₃N₉⁺: 682.0247; found 682.0212. [M+2H]⁺² Cald for C₂₄H₃₂Br₃N₉₂⁺. 341.5; found 341.5.

Tri-click 2-methyl-3-butyne-2-ol (TC-iPrOH; comparative example). To a solution of triazide (0.430 g, 1.5 mmol) in THF:H₂O (1:1, 15 mL), Na-L-ascorbate (10%, 0.45 mmol) was added followed by CuSO₄(1%, 0.045 mmol) under a nitrogen atmosphere. To this solution 2-methyl-3-butyne-2-ol (0.382 g, 0.44 mL, 4.52 mmol) was added dropwise. The reaction was stirred at 25° C. for 24 h. The resulting precipitate was isolated by vacuum filtration and washed with cold EDTA (0.1 M, 3×10 mL) and ice-cold water (3×10 mL). A white solid was recovered in good yield, (570 mg, 1.06 mmol, 71%). MP: 274-275° C. ¹H NMR (600 MHz, (CD₃)₂SO) δ: 7.65 (s, 3H), 5.66 (s, 6H), 5.08 (s, 3H), 2.44 (s, 9H), 1.42 (s, 18H). ¹³C NMR (151 MHz, (CD₃)₂SO) δ: 156.19, 139.61, 131.48, 120.44, 67.56, 48.67, 31.18, 16.87. Anal. Cal. for C₂₇H₃₉N₉O₃: C, 60.32; H, 7.31; N, 23.45. % Found: C, 58.62; H, 7.26; N, 21.71. IR (ATR, cm⁻¹): 3057, 1368, 1216, 1175, 1058, 955, 857,827, 806, 714. ESI-MS m/z: [M+Na]⁺ Cald for C₂₇H₃₉N₉O₃Na⁺: 560.3; found 560.3.

### Polyamine Controls

2,4,6-Tris-(aminomethyl)-mesitylene (T1; comparative example). To a solution of triazide (1.00 g, 2.1 mmol) in absolute EtOH (40 mL), Pd/C (10% w/w, 100 mg) was added and the reaction was stirred under a H₂ balloon for 6 h. It was then filtered through a small pad of celite, which was rinsed with EtOAc (3×15 mL). The filtrate was concentrated to afford triamine (1.186 g, quant.) as a white solid. MP: 149-150° C. ¹H NMR (600 MHz, CDCl₃) δ: 3.95 (s, 6H), 2.48 (s, 9H). ¹³C NMR (151 MHz, CDCl₃) δ: 138.13, 133.48, 40.84, 15.42. Melting point and ¹H NMR spectra were in agreement with literature data. ATR-IR (cm⁻¹): 3342, 2958, 2903, 2218, 1568, 1448, 1379, 1298, 1050, 861, 620. ESI-MS m/z: [M+H]⁺ Cald for C₁₂H₂₂N₃⁺: 208.2; found 208.2.

2,4,6-Tris-(1,2-ethanediamine)-mesitylene (T2; comparative example). To a solution of 2,4,6-tris-(bromomethyl)-mesitylene (1.200 g, 3.0 mmol) in dry THF (50 mL), ethylenediamine (8.04 mL, 120 mmol) was added. The resulting solution was stirred at 25° C. until complete conversion was observed by ¹H NMR (24 h). Solvent and excess diamine were removed under reduced pressure. The resulting oil was solubilized in MeOH (30 mL) and KOH (0.340 g, 6 mmol) was added and the inorganic salts were precipitated by the addition of diethyl ether and removed my filtration. The solvent was removed under reduced pressure and product dried under schlenk line. The resulting amine scaffold was identified as a thick oil (0.673 g, 1.99 mmol, 67%). ¹H NMR (600 MHz, CDCl₃) δ: 3.76 (s, 6H), 2.84-2.75 (m, 12H), 2.42 (s, 9H). ¹³C NMR (151 MHz, CDCl₃) δ: 135.03, 135.00, 52.84, 48.47, 41.68, 15.48. ¹H NMR was in agreement with literature data. ATR-IR (cm⁻¹): 3266, 2852, 1651, 1568, 1450, 1338, 1105, 1030, 814, 747. ESI-MS m/z: [M+H]⁺ Cald for C₁₈H₃₇N₆⁺: 337.3; found 337.3.

2,4,6-Tris-(1,3-propanediamine)-mesitylene (T3; comparative example). To a solution of 2,4,6-tris-(bromomethyl)-mesitylene (1.200 g, 3.0 mmol) in dry THF (50 mL) 1,3-diaminopropane (10.02 mL, 120 mmol) was added. The resulting solution was stirred at 25° C. until the complete conversion was observed by ¹H NMR (24 h). The product was worked up as previously stated and the resulting amine scaffold was identified as a thick oil (1.003 g, 2.65 mmol, 88%). ¹H NMR (600 MHz, CD₃OD) δ: 3.86 (s, 6H), 2.98 (t, J=7.1 Hz, 6H), 2.89 (t, J=7.0 Hz, 6H), 2.46 (s, 9H), 1.87 (p, J=7.0 Hz, 6H). ¹³C NMR (151 MHz, CD₃OD) δ: 137.15, 135.46, 40.56, 29.53, 16.46. The ¹H NMR spectra was in agreement with literature data. ATR-IR (cm⁻¹): 3256, 2922, 2856, 1567, 1452, 1377, 1327, 1103, 1026, 969, 815, 752. ESI-MS m/z: [M+H]⁺ Cald for C₂₁H₄₃N₆⁺: 379.3544; found 379.3542. [M+2H]⁺² Cald for C₂₁H₄₃N₆⁺²: 190.2; found 190.2. [M+3H]⁺³ Cald for C₂₁H₄₃N₆⁺³ Cald 127.1; found 127.1.

### DNA Recognition Procedures

Preparation of solutions. Tri-Click samples were initially prepared in DMF and further diluted in HEPES buffer (80 mM). Metal complexes of the Tri-Click series were prepared in-situ by co-incubating with copper(II) nitrate trihydrate, manganese (II) chloride tetrahydrate or zinc(II) acetate dihydrate for 30 min at 37° C. prior to DNA addition.

DNA nuclease and condensation studies. Reactions were carried out in 80 mM HEPES (pH 7.4) unless otherwise stated and followed the general procedure: a total volume of 20 μL with 25 mM NaCl, 400 ng pUC19 at varying concentrations of test compounds (0.25-500 μM) were incubated at 37° C. for either 90 min, 6 h, 12 h or 24 h. Reactions were quenched by adding 6× loading buffer (Fermentas) containing 10 mM Tris-HCl, 0.03% bromophenol blue, 0.03% xylene cyanole FF, 60% glycerol, 60 mM EDTA and samples were loaded onto an agarose gel (1.2%) containing 4 μL EtBr. Electrophoresis was completed at 70 V for 1 h in 1×TAE buffer. DNA studies conducted in the presence of reductant were supplemented with 1 mM Na-L-ascorbate and incubated at 37° C. for 30 min. Electrophoresis was carried out at 70 V for 90 minutes in 1×TAE buffer and photographed using a UV transilluminator.

### DNA Damage Studies of Cu(II)-TC1

Competitive ethidium bromide displacement assay. The DNA binding affinity of the tripodal series was determined over a 2 h time period using calf-thymus DNA and synthetic alternating co-polymers poly[d(A-T)₂)] and poly[d(G-C)₂] by ethidium bromide fluorescence quenching in a similar manner to the high throughput method previously reported by Kellett et al. (McCann, M., McGinley, J., Ni, K., O'Connor, M., Kavanagh, K., McKee, V., Colleran, J., Devereux, M., Gathergood, N., Barron, N. et al. (2013) A new phenanthroline-oxazine ligand: synthesis, coordination chemistry and atypical DNA binding interaction. Chem. Commun., 49, 2341-2343). Each drug concentration was measured in triplicate, on at least two separate occasions, and the apparent binding constants were calculated using Kₐₚₚ=Kₑ×12.6/C₅₀ where Kₑ=9.5×10⁶ M⁻¹.

Atomic Force Microscopy (AFM). AFM samples were prepared according to the general procedure detailed below in the presence and absence of exogenous reductant. In the presence of reductant: A total volume of 20 μL containing pUC19 (200 ng), MgCl₂ (10 mM), varying concentrations of test compound Cu(II)-TC1 (0.5, 1.0, 7.5 and 10 μM) in the presence of Na-L-ascorbate (1 mM) were incubated for 25 min at 37° C. Samples were further diluted to a DNA concentration of 2-3 ng/μL in a final volume of 10 μL with nuclease free H₂O, pipetted directly onto freshly cleaved mica and incubated for a further 5 min. In the absence of reductant: A total volume of 10 μL final concentrations of 3 ng/μL of pUC19 (NEB, N3041), 5 mM MgCl and varying concentrations of test compound Cu(II)-TC1 (5, 10 and 30 μM) samples were incubated at 37° C. for 1 h. Mica was freshly cleaved prior to incubating with samples (10 μL, 5 min). All samples were rinsed thoroughly with nuclease free water (500 μL) and dried under compressed air. AFM images were acquired in ambient air with a commercial microscope, in tapping-mode, using super sharp silicon cantilevers with a 40 N/m force constant. Topographic images were recorded at a scanning rate of >1 Hz, and a resonance frequency of about 300 kHz (nominal value). Images were processed using the WSxM software.

DNA cleavage in the presence of ROS scavengers. The assay was conducted according to reported methods (Fantoni, N. Z., Molphy, Z., Slator, C., Menounou, G., Toniolo, G., Mitrikas, G., McKee, V., Chatgilialoglu, C. and Kellett, A. (2019) Polypyridyl-Based Copper Phenanthrene Complexes: A New Type of Stabilized Artificial Chemical Nuclease. Chem. Eur. J., 25, 221-237; and, Slator, C., Molphy, Z., McKee, V. and Kellett, A. (2017) Triggering autophagic cell death with a di-manganese(II) developmental therapeutic. Redox Biol., 12, 150-161). Briefly, to a final volume of 20 μL, 80 mM HEPES, 25 mM NaCl, 1 mM Na-L-ascorbate, and 400 ng of pUC19 DNA were treated with varying drug concentrations Cu(II)-TC1 (0.5, 1.0, 2.5, and 4.0 μM) in the presence ROS scavengers; sodium azide (NaN₃, 10 mM), 4,5-dihydroxy-1,3-benzenedisulfonic acid (tiron, 10 mM), D-mannitol (10 mM), N,N′-dimethylthiourea (DMTU, 10 mM), and L-methionine (10 mM). Reactions were vortexed and incubated at 37° C. for 30 min and electrophoresis was carried out at 70 V for 1 h in 1×TAE.

DNA cleavage in the presence of repair enzymes. Supercoiled pUC19 DNA (400 ng) in 4 mM HEPES, 25 mM NaCl, 1 mM Na-L-ascorbate in a final volume of 20 μL nuclease free H₂O, was pre-incubated with Cu(II)-TC1 for 30 min at 37° C. at 2.5, 5.0 and 7.5 μM. The following reactions were supplemented with associated buffers as per manufacturer's recommendations. Repair enzymes Fpg, endonuclease (Endo) III, Endo IV, Endo V and hAAG (2 units) were added to the reaction mixture and incubated for 30 min at 37° C. Samples were denatured with 0.25% SDS, 250 μg/mL proteinase K and heated to 50° C. for 20 min. Reactions were quenched by 6× loading buffer, loaded onto agarose gels and subjected to electrophoresis as stated above.

### Intracellular DNA Damage

Blood Sample Collection. Blood samples from healthy volunteers without having any pathology was collected from the Hematology Lab at the Clinical Chemistry Department at Sahlgrenska University Hospital, Gothenburg, Sweden. Gradient centrifugation using Lymphoprep (Axis-Shield PoC AS, Oslo, Norway) was used to collect peripheral mononuclear blood cells (PBMCs).

Treatment of PBMCs with TC1 and copper (II) nitrate trihydrate. 100 mM stock solutions of TC1 and copper (II) nitrate trihydrate was prepared in DMSO and Milli Q® (MQ) water respectively and stored at −20° C. until further use. Co-incubation of TC1 at three different concentrations, 75 μM, 100 μM and 300 μM with a Cu(II) concentration of 300 μM was performed for 30 min at 37° C. in 1×RPMI. 5×10⁵ PBMCs were introduced to the Cu(II)-TC1 complex and incubated for 1 h on a thermal block at 37° C. Final DMSO concentrations did not exceed 0.1% v/v.

Treatment of PBMCs with antioxidants. 100 mM stock solution of sodium pyruvate, tiron, L-histidine, and D-mannitol was prepared in MQ water. Cells were pre-treated with 1 mM scavengers for 2 h prior to Cu(II)-TC1 exposure.

Extraction of DNA. After drug treatment and antioxidant studies, DNA was extracted using GenElute-Mammalian Genomic DNA Miniprep Kit and eluted in 10 mM Tris-Cl (pH 8.5). DNA concentrations were measured using a NanoDrop 1000 spectrophotometer. Care was taken to avoid loss of genomic integrity by using wide-bore tips.

Fluorescent labelling of Cu(II)-TC1 induced DNA damage. DNA (100 ng) was incubated with APE1, Fpg or Endo III (2.5 U) in 1×CutSmart Buffer for 1 h at 37° C. Samples pre-treated with scavengers were incubated with a mixture of enzymes. The in vitro DNA repair was followed by 1 h incubation at 20° C. with dNTPs (1 μM of dATP, dGTP, dCTP, 0.1 μM dTTP and 0.1 μM Aminoallyl-dUTP-ATTO-647N, 1×NEBuffer 2 and DNA polymerase 1 (2.5 U) at 20° C. Subsequently, the reaction was terminated with 2.5 μL of 0.25 M EDTA.

Silanization of coverslips. Two silane molecules (3-aminopropyl) triethoxysilane (APTES), allyltrimethoxysilane (ATMS), and acetone was used to silanize standard 22×22 mm coverslips. They were carefully put into the solution consisting of acetone (1% APTES, 1% ATMS, v/v) and coated for 1 h. Coated coverslips were rinsed with acetone and MQ water to remove residues. The air dried silanized coverslips were stored in a parafilm sealed pertri dishes and used within a week.

Staining and Instrument. The Aminoallyl-dUTP-ATTO-647N labelled DNA samples were diluted with 0.5×TBE and were stained with 320 nM YOYO-1 (Invitrogen). 2 μL β-mercaptoethanol was added in total volume of 100 μL. The DNA samples were extended by placing the 3.8 μL solution at the interface of the silanized coverslip and a microscopy slide (VWR Frosted). Zeiss Observer. Z1, equipped with an Andor iXON Ultra EMCCD camera and a Colibri 7 LED illumination system was used to obtain the fluorescence images. Each image consisted of two colours, YOYO-1 (green), and aminoallyl-dUTP-ATTO-647N (red) using appropriate filters band-pass excitation filters (475/40 and 640/30 nm) and bandpass emission filters (530/50 and 690/50 nm), respectively with exposure times of 10 ms and 500 ms.

### Software Analysis

AFM images were processed using the WSxM software to remove the background slope and normalize the z-scale across all images, no additional filtering was performed. Microscopy images were analysed using a custom-made software. The total number of colocalized aminoallyl-dUTP-ATTO-647N labels in an image set was divided by the total DNA length in pixels to get the ratio as DNA damage/length. The final values were depicted as Dots/MBp. It was calculated by stretching lambda DNA molecules (48,502 base pairs (bp)) in a similar buffer conditions and we observed that 1 μm=˜3000 bp. Band densitometry was quantified using the Image J software package were each reaction was carried out in triplicate.

### Statistics

Intracellular DNA damage and band densitometry studies were performed in triplicate and analysed in GraphPad Prism using un-paired T tests. The data was considered statistically significant with *P≤0.05; **P≤0.01; ***P≤0.001.

### Screening for Innate DNA Binding Interactions

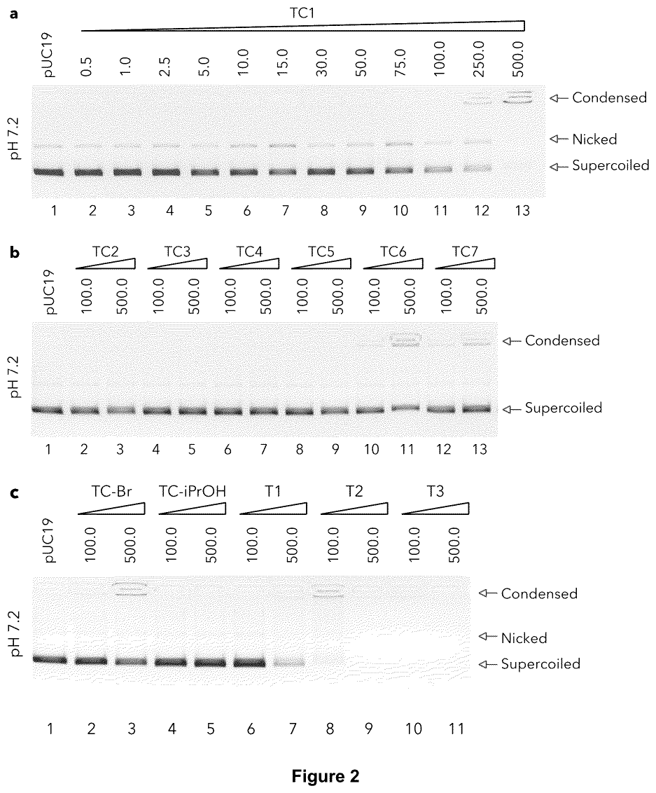

The DNA recognition properties of the TC series, along with the control compounds, were probed using electrophoresis experiments with supercoiled pUC19 DNA. TC1 was examined across a wide concentration range and found to condense DNA at high concentrations between 250 and 500 μM (FIG. 2a, lanes 11-13). TC2-7 (inclusive) were then examined at high loading (100 and 500 μM) with slight aggregation effects observed for TC6 and TC7 (FIG. 2b, lanes 10-13). The control agent TC-Br had only a mild condensation effect at 500 μM while TC-iPrOH had no effect on pUC19 (FIG. 2c). The tripodal amine controls were more active at condensing DNA; T1 was observed to mildly condense pUC19 while T2 and T3 were highly active at 100 μM which was not surprising since both agents can carry a 6+ cationic change and are flexible. Taken together, these results indicate that electrostatic interactions can play a role in DNA aggregation but the TC series are not potent DNA condensation agents compared to simple polyamines or C₃-symmetric opioids.

### Nuclease Activity in the Presence of First Row Metal Ions

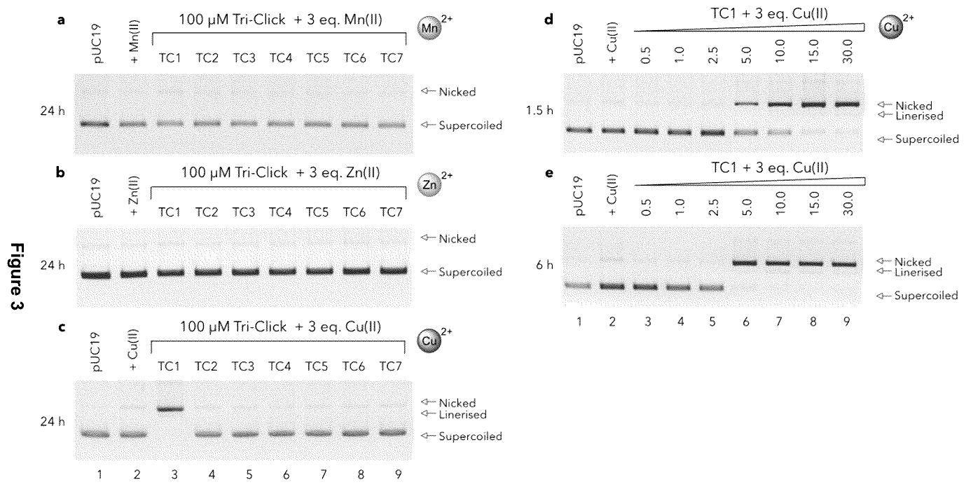

The interactions of the TC series was next examined in the presence of first row metal ions. Tri-Click scaffolds were pre-incubated with three molar equivalents of either Cu(II), Mn(II) or Zn(II) ions-all of which are known to catalyse DNA strand scission when complexed to specific ligands-prior to incubation with DNA for a period of 24 h at pH 7.2 (FIG. 3a-c). The 3:1 Cu(II):TC1 complex (hereafter referred to as Cu(II)-TC1) displayed DNA damaging properties (FIG. 3c). Here, supercoiled pUC19 was completely relaxed to its open circular form (OC) indicating the formation of single strand breaks (SSBs).

Since these reactions were conducted in the absence of exogenous reagents (i.e. added reductant or peroxide) the Cu(II)-TC1 complex appears capable of ‘self-activating’ chemical nuclease activity—an effect previously observed for several polynuclear Cu(II) complexes and mononuclear Cu(II) complexes of marine alkaloids.

To establish the role of am(m)ine coordination in SSB formation, the reactions of TC ligands with Cu(II) were repeated in acidic buffer (pH=4.0). In these conditions the chemical nuclease activity of Cu(II)-TC1 was negligible indicating that metal ion binding, and thus chemical nuclease activity, is inhibited due to the protonation of each primary am(m)ine site in TC1. Finally, the activity of Cu(II)-TC1 was monitored at lower concentrations and shorter timeframes (again in the absence of exogenous reagents) at neutral pH. After 90 min, the presence of 5 μM of Cu(II)-TC1 initiates SSB formation with complete conversion to the nicked form at 50 μM exposure (FIG. 3d). Extending the incubation time to 6 h then showed more efficient DNA cleavage could be obtained with 5 μM of TC1 (FIG. 3e).

### DNA Binding and Time-Course Cleavage Interactions

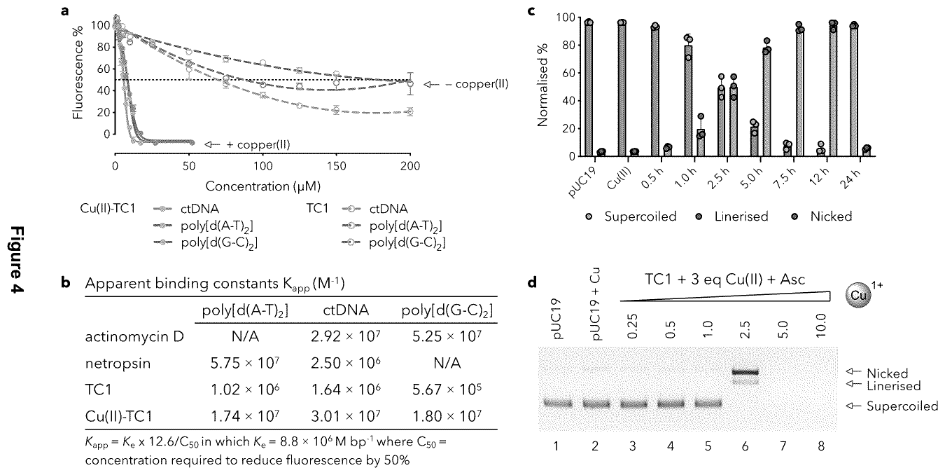

To establish how copper(II) binding influences DNA recognition, apparent DNA binding studies were conducted with calf-thymus DNA (ctDNA) along with alternating co-polymers poly[d(A-T)₂] and poly[d(G-C)₂]. These experiments determine the indirect binding constant based on the ejection of bound ethidium bromide (EtBr), which serves as an intercalating reporter. The TC1 ligand demonstrated moderate DNA binding constants of ˜10⁶ M⁻¹ with ctDNA and A-T duplex polymers while a lower value of ˜10⁵ M⁻¹ was observed with poly[d(G-C)₂]. However, in the presence of three copper(II) nitrate ions, binding increased by 10- and 100-fold (˜10⁷ M⁻¹) in all three of the duplexes (FIGS. 4a and b). Although the binding constants of Cu(II)-TC1 did not show specificity for A-T or G-C rich DNA, they were nonetheless comparable to the high-affinity intercalator actinomycin D and the minor groove binder netropsin.

DNA cleavage by Cu(II)-TC1 was mapped using a time-course study (30 min to 24 h) with the Cu(II)-TC1 (25 μM) in the absence of reductant (FIG. 4c). Band densitometry analysis showed almost 50% SSB formation after 2.5 h and >95% of the plasmid was nicked by 7.5 h. The appearance of double strand breaks (DSBs) could be detected after 24 h at limited concentration (˜5%) and after the appearance of SSBs indicating they originate from two independent nicking events. In the absence of reductant, Cu(II)-TC1 clearly produces SSBs; to better understand how this complex might behave biologically, the addition of endogenous reducing agents were employed to mimic physiological conditions. Here, strand scission was catalysed in the presence of the natural reductant, Na-L-ascorbate, where SSBs and DSBs occur at low concentration (2.5 μM of TC1) and short reaction times (30 min, FIG. 4d), thereby suggesting an oxidative cleavage mechanism is at play.

### Atomic Force Microscopy (AFM) Analysis of DNA Damage

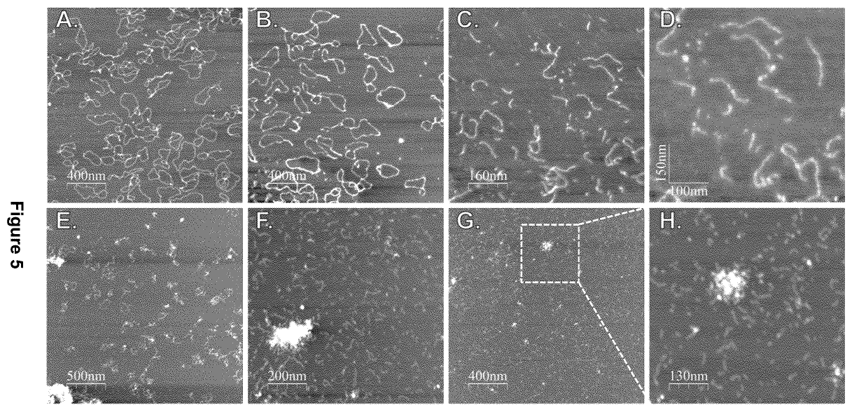

Using gel electrophoresis the present inventors have shown that Cu(II)-TC1 is capable of cleaving supercoiled plasmid DNA to open circular and linear forms via SSB and DSB formation. To study this conformational change in more detail, AFM measurements were undertaken with pUC19 exposed to Cu(II)-TC1. In the presence of excessive reductant, DNA remained predominantly in its supercoiled state, however the addition of 500 nM of Cu(II)-TC1 initiated single strand breaks that were visualised (Figure Sa). Points where ligand-DNA binding occur were identified as increases in the height profile of circular structures (FIG. 5b). An increase in complex concentration (1.0 μM) rendered plasmid DNA predominantly in its open circular form (FIG. 5b) with complete degradation reached at 7.5-10 μM exposure (FIGS. 5c and d). DNA damage was also visualised in the absence of reductant at a low concentration (5 μM) of the complex (FIG. 5e). Small compact clusters of fragmented DNA were intermediately observed and these small aggregates are evident in FIG. 5f-g where the complex concentration (again in the absence of reductant) was increased to 10 and 30 μM, respectively. On closer inspection of the cluster present in FIG. 5g, it can be clearly seen that small portions of fragmented DNA surround the compact particle (FIG. 5h). These compacted DNA fragments could not be visualised through gel electrophoresis due to their small size, however AFM analysis provides evidence of this interaction by the moderate condensation effects of the complex.

Probing the DNA Cleavage Mechanism with Radical Scavengers and BER Enzymes

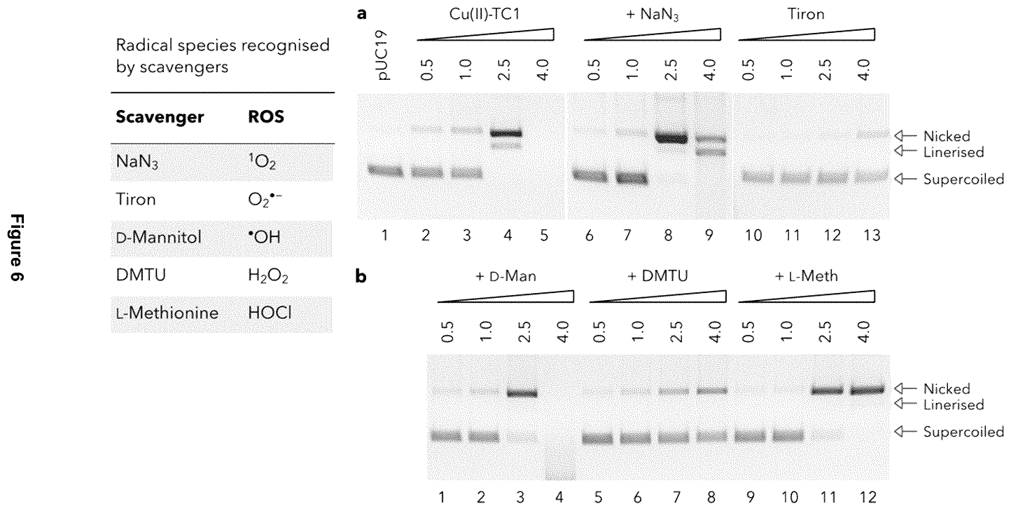

The Cu(II)-TC1 DNA damage mechanism was probed in vitro, where the role of radical species were analysed with ROS-specific scavengers and spin-trapping agents: NaN₃, tiron, D-mannitol, N,N′-dimethylthiourea (DMTU) and L-methionine. These antioxidants can sequester singlet oxygen (¹O₂), superoxide (O₂·⁻), the hydroxyl radical (·OH), hydrogen peroxide (H₂O₂) and hypochlorous acid (HOCl) or a combination thereof (see inset table FIG. 6). In these experiments, the delayed onset of SSB formation and the inhibition of DSBs were evident (FIG. 6). H₂O₂ was identified as the most prevalent radical species required in the cleavage mechanism as DMTU (N,N′-dimethylthiourea) inhibited the complete transformation of supercoiled DNA to nicked open circular DNA.

The ·OH scavenging agent D-mannitol had almost no influence on DNA cleavage; L-methionine inhibited the formation of DSBs which may be attributed to scavenging of H₂O₂. Finally, the superoxide radical also appears to be important in the DNA cleavage mechanism since the introduction of tiron prevented DSB formation and delayed the onset of SSBs.

Without wishing to be bound by theory, these results indicate the DNA damaging mechanism by Cu(II)-TC1 does not follow classic Fenton chemistry (which is dependent on diffusible ·OH radical generation), but instead follows a superoxide dismutase (SOD) type mechanism, whereby electron transfer from a reduced Cu(I) centre promotes the formation of O₂·⁻ (or a metal-superoxo species), which is then converted to H₂O₂ by a second Cu(I) metal centre. Therefore, it might be the case that O₂·⁻ does not promote DNA damage directly, but that tiron simply impedes the downstream formation of the peroxide (or metal-peroxo Cu(II)—OOH type) cleavage species.

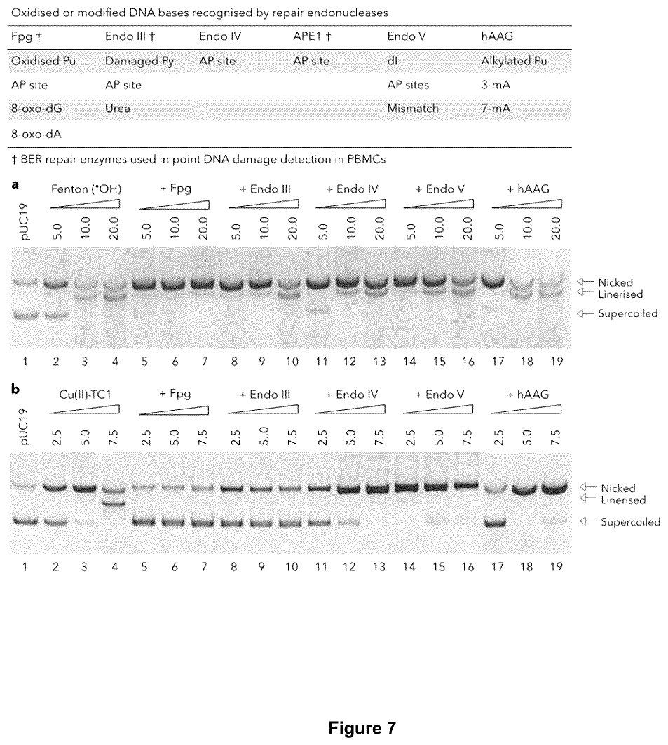

In order to examine the oxidative damage mechanism in greater detail, base excision repair (BER) enzymes, which can recognise specific lesion-specific modifications associated with oxidative damage were employed. Repair proteins that recognise base-specific DNA lesions—or a small a small set of lesions with a common structural motif—are known as glycosylases and carefully remove their cognate damaged bases to yield abasic sites. Specific repair endonucleases can then recognise these AP (apurine/apyrimidine) sites and mediate strand nicking adjacent to the base-free lesion to create a single nucleotide gap that is filled by the insertion of a new base. The BER and endonuclease enzymes employed in this study are listed in FIG. 7 (table inset) and initial experiments involved treating pUC19 with each enzyme to ensure that the plasmid was free of existing lesions.

Experiments were then designed to initiate DNA cleavage using a control hydroxyl radical (OH) generated from a Fenton Cu²⁺/H₂O₂ reaction alongside the Cu(II)-TC1 complex. After 30 min of continuous exposure to the complex (used to initiate DNA damage), specific BER enzymes were introduced and the reactions were allowed to incubate for a further 30 min with results compared to control experiments lacking repair enzymes. In the copper-mediated Fenton reaction, DNA damage was inhibited Fpg and Endo III, which indicates strand breaks are mediated by oxidised purine and pyrimidine bases generated by the hydroxyl radical (FIG. 7a). Reaction conditions for the Cu(II)-TC1 (2.5-7.5 μM) were optimised to initiate SSB and DSB cleavage of supercoiled pUC19 (FIG. 7b, lanes 2-4). In all cases, the presence of specific BER enzymes inhibited DSB formation since no linearised DNA was detected. In the presence of Fpg and Endo III, a significant reduction in SSB formation occurred thereby providing protection of intact plasmid DNA.

In conjunction with the inhibition profile of Endo IV (and to a lesser extend Endo V), these results indicate the Cu(II)-TC1 complex can oxidise both purine and pyrimidine bases and promote the formation of abasic sites.

### Selectivity of APE1 in Repairing Cu(II)-TC1 Mediated Single-Strand Breaks in PBMCs

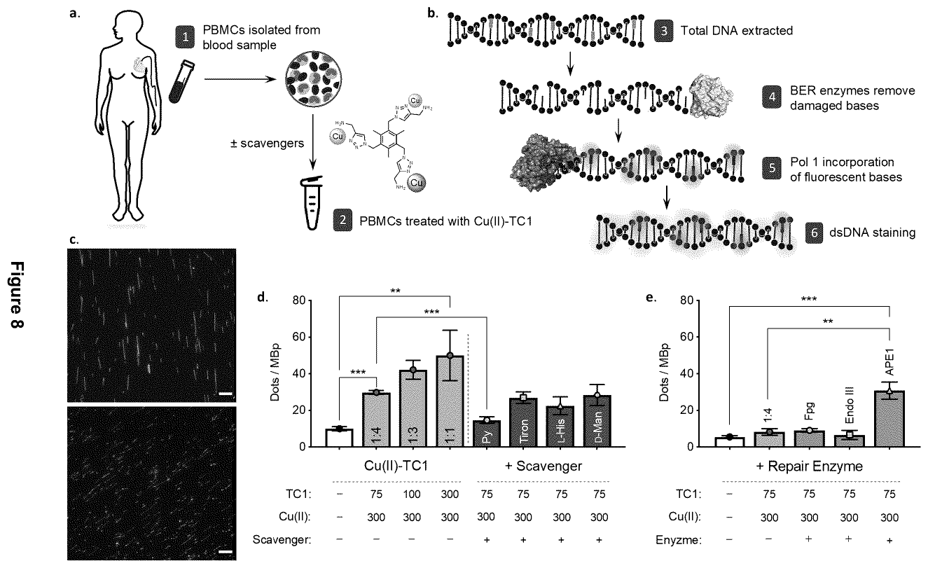

Intracellular DNA damage mediated by Cu(II)-TC1 was examined using peripheral blood mononuclear cells (PBMCs). An assay was designed (FIGS. 8a and b) whereby DNA extracted from metal complex-treated cells was initially exposed to specific BER enzymes and in a second step, DNA polymerase 1 was introduced with a fluorescent dNTP (aminoallyl-dUTP-ATTO-647N). Later, a dye (YOYO-1) is used to stain the DNA backbone and individual DNA molecules are stretched on silanized glass coverslips, where image analysis determines the extent of DNA damage by quantifying the incorporation of repaired bases on the single DNA molecule level (cf. FIG. 8c).

PBMCs were exposed to Cu(II)-TC1 and intracellular SSBs were quantified (FIG. 8d). In these experiments, the concentration of Cu(II) was kept constant (300 μM) while the TC1 concentration was varied to examine TC1:Cu(II) ratios from 1:1-1:4. Compared to untreated control cells, SSB formation increased ˜5.0 fold as the concentration of Cu(II)-TC1 increased to 300 μM. Next, PBMCs were incubated prophylactically with specific antioxidants-sodium pyruvate (H₂O₂), tiron (O₂·⁻), L-histidine (¹O₂), or D-mannitol (·OH)—prior to the introduction of the Cu(II)-TC1 complex (FIG. 8d). With sodium pyruvate, the decrease in Cu(II)-TC1-mediated SSBs was highest indicating that H₂O₂ is required in the DNA cleavage mechanism, in agreement with the in vitro experiments. Overall, the inhibition of SSB formation follows the trend: sodium pyruvate>>L-histidine>tiron>D-mannitol, indicating again that superoxide is not primary to SSB formation. Next, three different BER enzymes, Fpg, Endo III, and APE1 (derived from an AP endonuclease family similar to Endo IV) were employed to quantify the selective labelling of SSB repair. In PBMCs treated with Cu(II)-TC1, a SSB fold increase of ˜1.1 (FpG), ˜0.8 (Endo III) and ˜3.7 (APE1) was detected, when compared to DNA polymerase 1 treated Cu(II)-TC1 samples (FIG. 8e). That APE1 was the most efficient enzyme in detecting the SSBs indicates that the majority of lesions generated by this complex are apurinic/apyrimidinic sites. These results overlap with in vitro analysis and demonstrate abasic sites generated by a TC1-Cu-peroxide mediated mechanism leading to the formation of intracellular SSBs.

### CONCLUSION

The discovery of new metallodrugs with alternative modes of action is important not only to overcome resistance factors, but also in treating recalcitrant cancers where no (or severely limited) treatment options remain. The present inventors identified a click chemistry-based strategy to produce bioactive polynuclear complexes. A series of alkyl and aromatic amines with alkyne groups were developed and clicked to a 1,3,5-triazide mesitylene core to produce a library of polyamine ligands referred to as the Tri-Click (TC) series.

Click chemistry has not previously been widely employed to design small molecule metallodrugs with nucleic acid recognition properties. TC1 was identified as a copper(II) binding ligand with potent chemical nuclease activity. TC1 was produced by reacting a primary amine, propargylamine, with mesitylene triazide and it appears this specific combination is required for copper-mediated nuclease activity.

TC1 contains N,N donors that are capable of forming a 5-membered ring with copper (one from the primary amine and a second from the 3′ imine of the triazole ring). Coordination of copper (or other metal ions, including Cu²⁺, Cu¹⁺, Co¹⁺, Co²⁺, Fe²⁺, Fe³⁺, Mn²⁺, Zn²⁺, Pt²⁺, Pt⁴⁺ and Ru²⁺) is beneficial for DNA binding and oxidation since protonation of the am(m)ine group in acidic buffer (pH 4.0) provides no associated cleavage activity.

The binding affinity of TC1 to duplex DNA is moderate (10⁵-10⁶ M⁻¹) but in the presence of titrated copper(II) activity increases to 10⁷ M⁻¹ for calf thymus and AT and GC co-polymers. This affinity places it in line with classical DNA recognition agents such as netropsin, dactinomycin and several state-of-art intercalating metallodrugs but it appears Cu(II)-TC1 does not purely interact by groove binding or intercalation. Subsequent AFM analysis of Cu(II)-TC1 with plasmid DNA in the absence of reductant demonstrated the emergence of compact clusters surrounded by small portions of fragmented DNA. Based on this evidence it appears Cu-TC1 interacts electrostatically with DNA and initiates condensation that involves aggregation of sheared nucleic acid fragments that arise from SSBs. In a broadly similar vein, phosphate clamping polynuclear platinum complexes such as TriplatinNC which contain three trans-symmetric Pt(II) ions—and which also carry a net +6 cationic charge—demonstrate high DNA binding affinities and are potent condensation agents of DNA duplexes and nucleic acids in general.

Cu(II)-TC1 demonstrates ‘self-activating’ DNA cleavage and is capable of mediating single strand breaks in the absence of an external (exogenous) reducing agent. In the presence of added ascorbate, the Cu(I)-TC1 complex is generated and is a potent DNA oxidant inducing SSBs and DSBs (arising from proximate SSBs or clustered damage) at low micromolar concentrations. This enhanced activity was corroborated by AFM imaging in the presence of ascorbate where SSBs and DSBs were identified across a 0.5-10 μM concentration gradient. DNA damage assays, performed both in vitro and in peripheral blood mononuclear cells (PBMCs), were then applied to probe the mechanism of copper-mediated DNA damage.

Several conclusions can be drawn by comparing in vitro quenching data and results from intracellular trapping experiments (where available) to other ‘self-activating’ copper complexes. In contrast to naturally occurring systems, strand breaks were completely inhibited in vitro by sequestering superoxide with tiron. SSBs and DSBs were attenuated in the presence of pyruvate. DNA cleavage is not affected by hydrogen peroxide scavengers. Instead, the complex mediates intracellular singlet oxygen production in its doubly reduced form (i.e. where both copper ions are in the +1 oxidation state). Considering these classes of ‘self-activated’ systems, the DNA damaging activity of Cu(II)-TC1 appears closely related to the copper(II) complexes of polypyrrole-based alkaloids and macrocyclic colibactin since: a.) activity is dependent on hydrogen peroxide; b.) the superoxide radical is not involved during the cellular cleavage process, and; c.) singlet oxygen and Fenton-type products are not involved in the DNA damage mechanism.

The present inventors have identified and characterised new click chemistry methods to produce novel polynuclear DNA damaging metallodrugs. The present inventors demonstrated that at least TC1 from this series directly mediates oxidative DNA damage in the presence of copper(II) using a ‘self-activating’ mechanism. Therefore, the example compounds are useful in the treatment and detection of cancer. There is good agreement between the in vitro and cellular DNA damaging modes supporting a mechanism closely related to copper complexes of naturally occurring marine alkaloids along with macrocyclic colibactin. Therefore, the present inventors have prepared new compounds which simulate, or improve on, DNA damaging natural products. These discoveries provide enhanced DNA damaging agents with unique chemotherapeutic properties that may circumvent innate cellular DNA repair machinery.

The present inventors believe that the presently claimed compounds of Formula (I) and Formula (II) can be conjugated to vectors directed to specific genes. Such conjugates can be used in gene therapy for both the detection and elimination of aberrant genetic elements via metal ion mediated oxidative damage or direct binding through covalent or non-covalent mechanisms.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Although certain example embodiments of the invention have been described, the scope of the appended claims is not intended to be limited solely to these embodiments. The claims are to be construed literally, purposively, and/or to encompass equivalents.

1. A compound of Formula (II):
M_{z}— Formula (I)
(II)
or a pharmaceutically acceptable salt thereof, wherein:
Formula (I) is a compound of structure 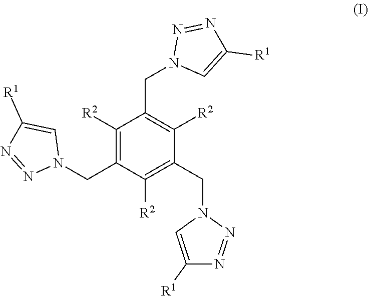
or a pharmaceutically acceptable salt thereof wherein:
R¹ is —CH₂OH, —C(═O)OH, —C(═O)NH₂, —CH₂NH₂, —CH₂NHCH₃, —CH₂N(CH₃)₂, meta-C₆H₄NH₂, meta-C₆H₄NHCH₃, meta-C₆H₄N(CH₃)₂,para-C₆H₄N(CH₃)₂, 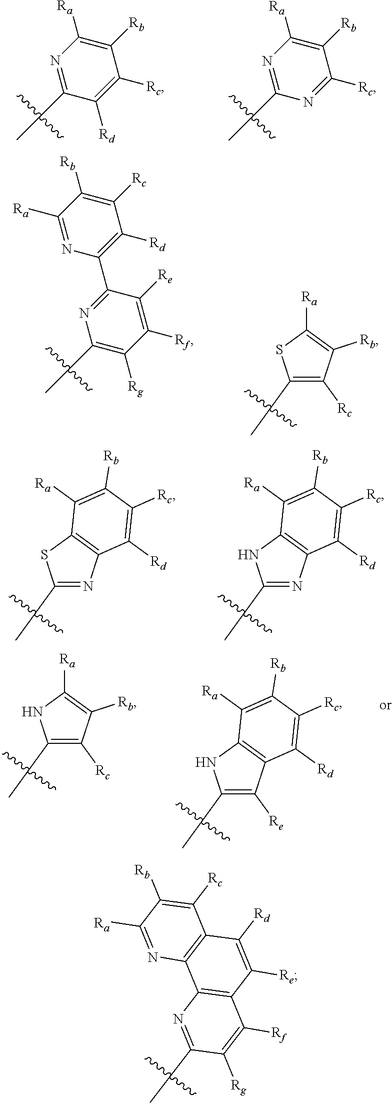
Wherein: R^{a} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{b} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{c} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{d} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{e} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{f} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃,
R^{g} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R² at each occurrence is selected from the group consisting of: —CH₃, H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
M is a metal cation coordinated or chelated to the compound of Formula (I), in which M is selected from the group consisting of Cu²⁺, Cu¹⁺, Mn²⁺, Zn²⁺, Pt²⁺, Pt⁴⁺, Ru²⁺, Ru³⁺, Co¹⁺, Co²⁺, Fe²⁺ and Fe³⁺; wherein for each M the metal cation is any isotope of the metal cation; and
z is an integer.

2. The compound of claim 1 wherein Formula (I) is selected from the group consisting of:

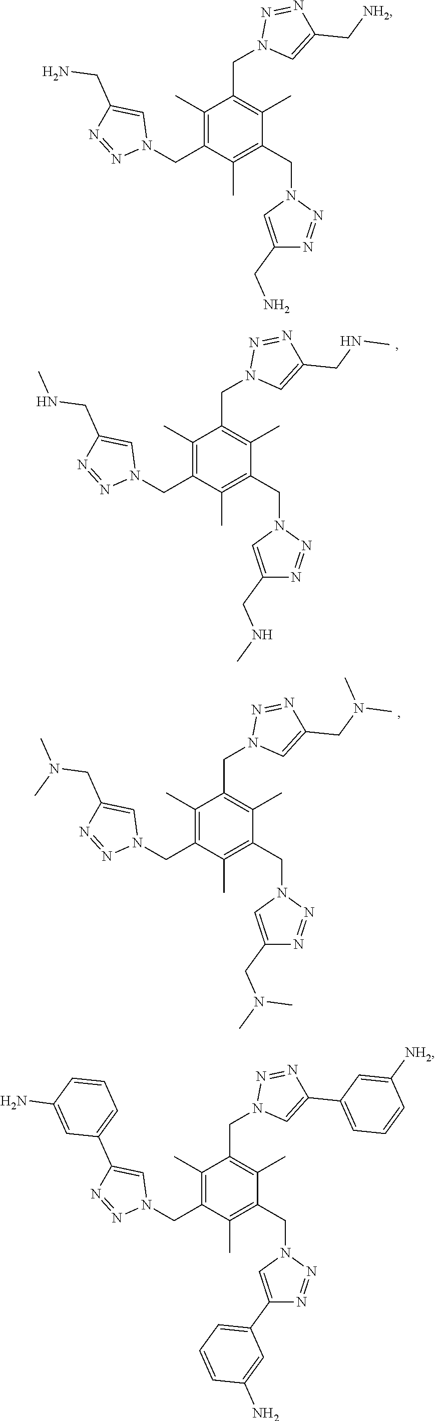

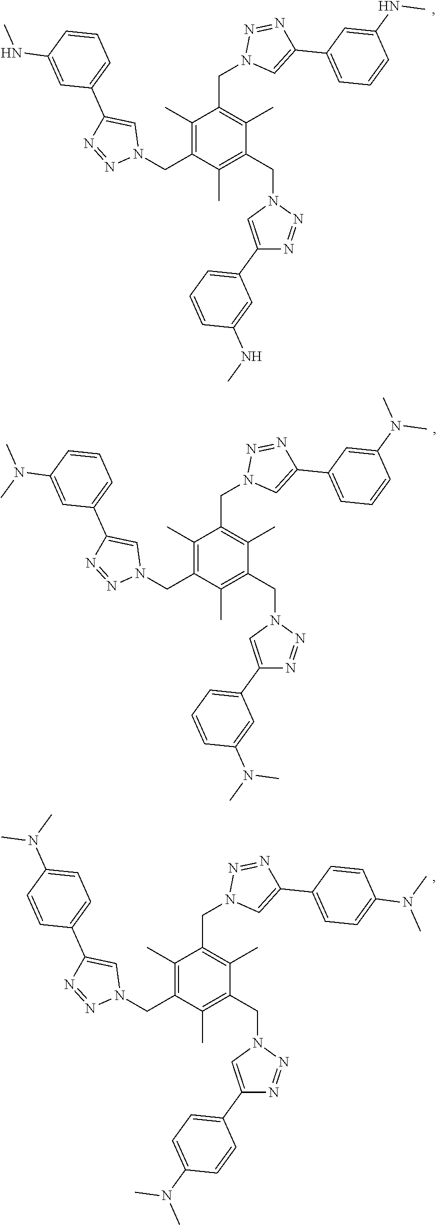

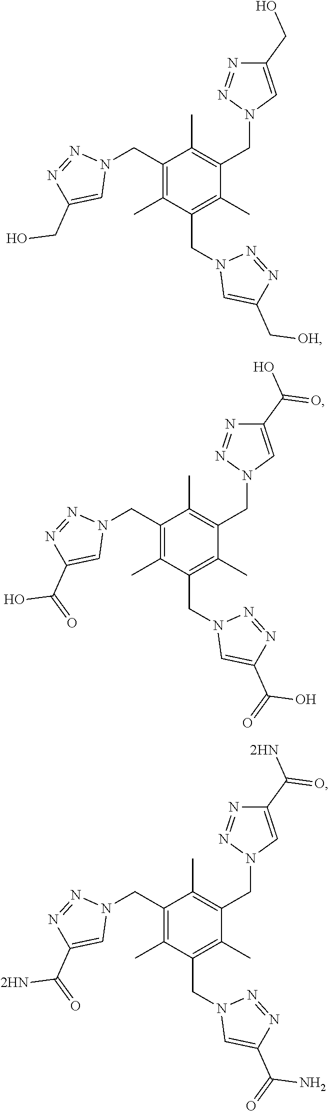

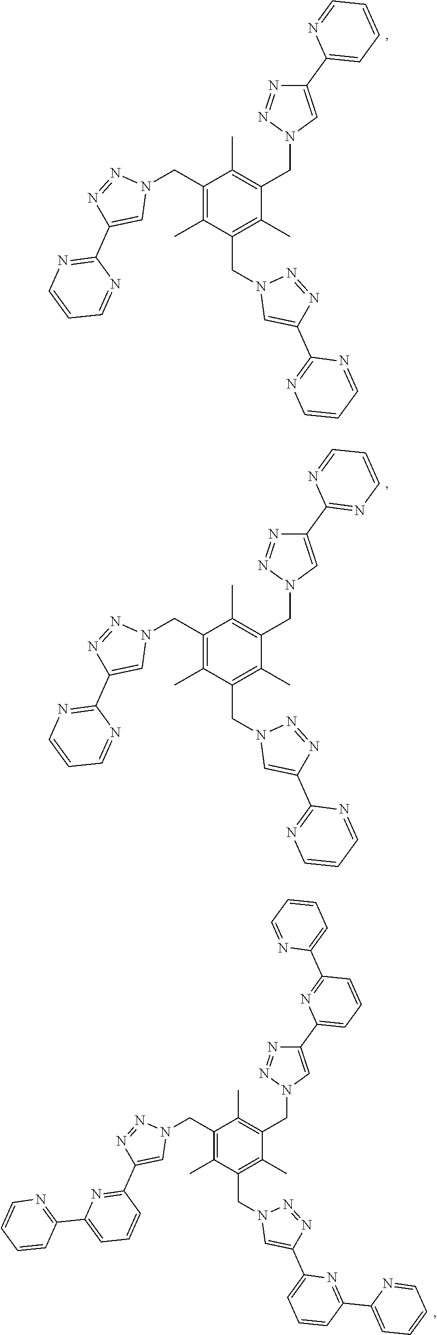

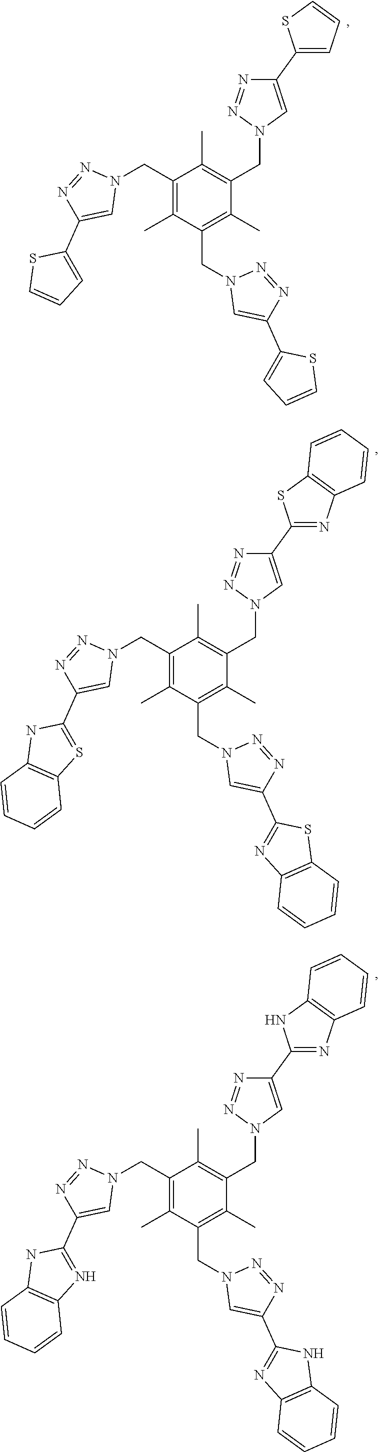

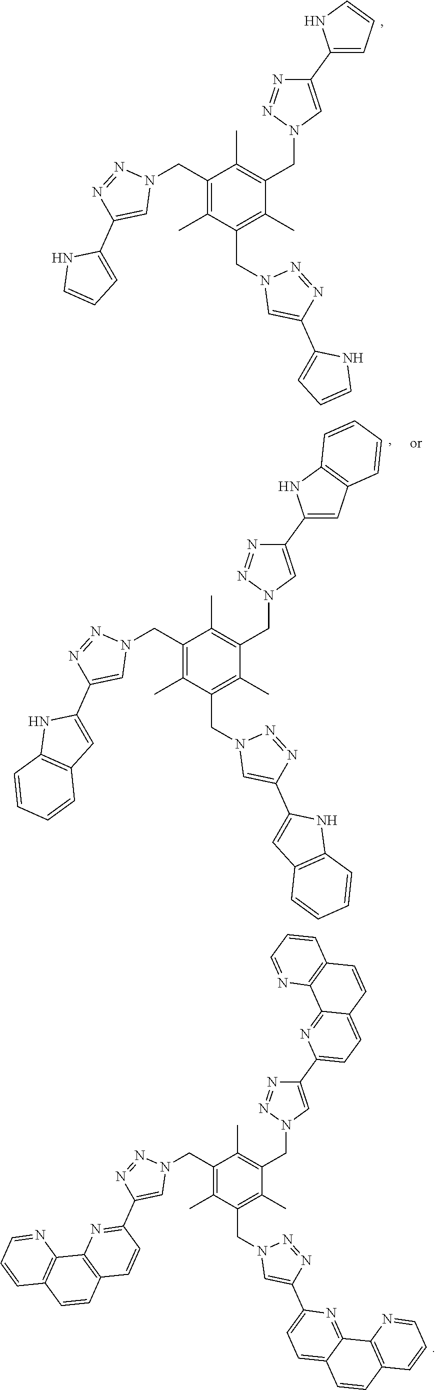

3. The compound of claim 1, wherein z is selected from the group consisting of: 1, 2, 3, 4, 5 or 6.

4. The compound of claim 1, wherein z is 3.

5. The compound of claim 1, wherein M is Cu²⁺.

6. The compound of claim 1, wherein the compound is a compound of Formula (II-A): 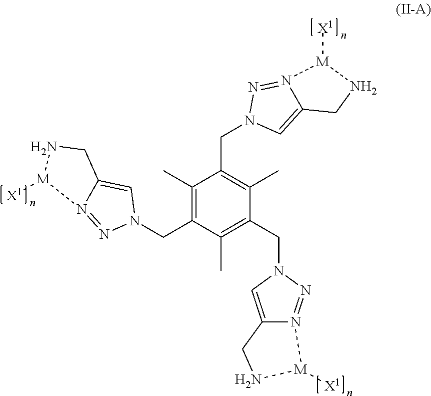
wherein:
M is Cu²⁺, Cu¹⁺, Mn²⁺, Zn²⁺, Pt²⁺, Pt⁴⁺, Ru²⁺, Ru¹⁺, Co¹⁺, Co²⁺, Fe²⁺ or Fe³⁺;
X¹ is a counter ion or a water molecule or a ligand that binds to M; and,
n is an integer selected from the group consisting of: 1, 2, 3, 4, 5 or 6.

7. The compound of a claim 1, wherein the compound is a compound of Formula (II-B): 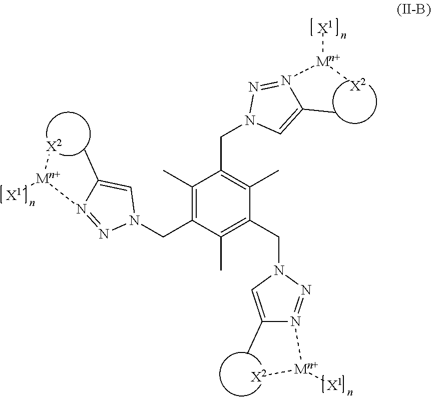
wherein:
M is Cu²⁺, Cu¹⁺, Mn²⁺, Zn²⁺, Pt²⁺, Pt⁴⁺, Ru²⁺, Ru³⁺, Co¹⁺, Co²⁺, Fe²⁺ or Fe³⁺;
X¹ is a counter ion or a water molecule or a ligand that binds to M;
n is an integer selected from the group consisting of: 1, 2, 3, 4, 5 or 6; and
X² is a heteroatom within a cyclic substituent (optionally a cycloalkyl group including a N atom) and X² is coordinated to the metal ion M.

8-11. (canceled)

12. A method of forming a compound of Formula (II):
M_{z}— Formula (I)
(II)
or a pharmaceutically acceptable salt thereof, wherein:
Formula (I) is a compound of structure 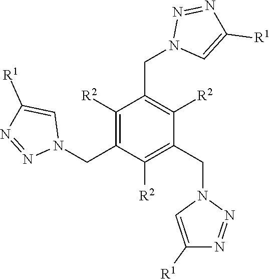
or a pharmaceutically acceptable salt thereof wherein:
R¹ is —CH₂OH, —C(═O)OH, —C(═O)NH₂, —CH₂NH₂, —CH₂NHCH₃, —CH₂N(CH₃)₂, meta-C₆H₄NH₂, meta-C₆H₄NHCH₃, meta-C₆H₄N(CH₃)₂,para-C₆H₄N(CH₃)₂, 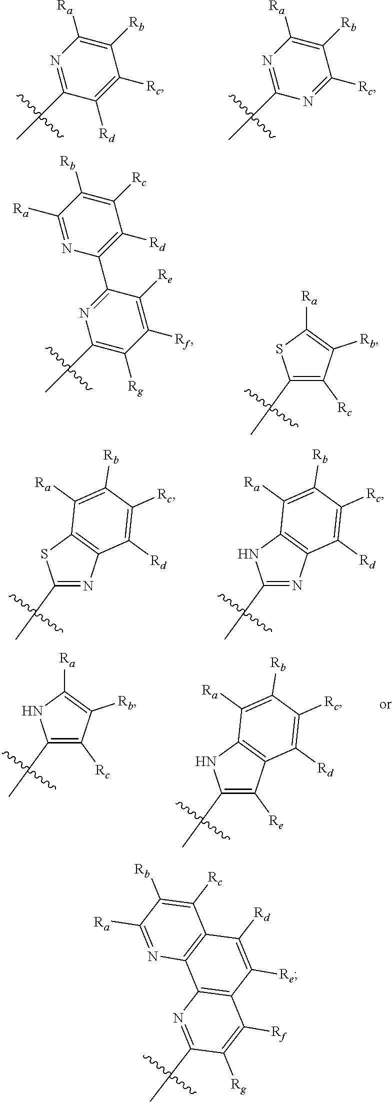
Wherein: R^{a} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{b} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{c} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{d} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{e} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{f} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃,
R^{g} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R² at each occurrence is selected from the group consisting of: —CH₃, H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
M is a metal cation coordinated or chelated to the compound of Formula (I), in which M is selected from the group consisting of Cu²⁺, Cu¹⁺, Mn²⁺, Zn²⁺, Pt²⁺, Pt⁴⁺, Ru²⁺, Ru³⁺, Co¹⁺, Co²⁺, Fe²⁺ or Fe³⁺; wherein for each M the metal cation is any isotope of the metal cation; and
z is an integer;
wherein the method comprises the steps of:
reacting a compound of Formula (I) 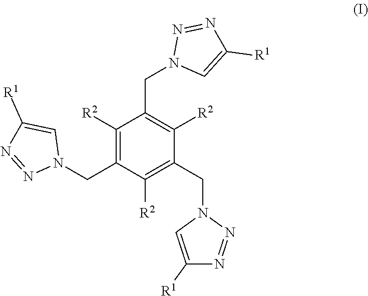
with a metal cation M.

13. The method of claim 12, wherein the metal cation M is Cu²⁺.

14. A compound of Formula (I): 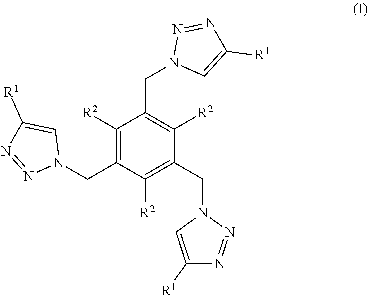
or a pharmaceutically acceptable salt thereof, wherein:
R¹ is —CH₂OH, —C(═O)OH, —C(═O)NH₂, —CH₂NH₂, —CH₂NHCH₃, —CH₂N(CH₃)₂, meta-C₆H₄NH₂, meta-C₆H₄NHCH₃, meta-C₆H₄N(CH₃)₂, para-C₆H₄N(CH₃)₂, 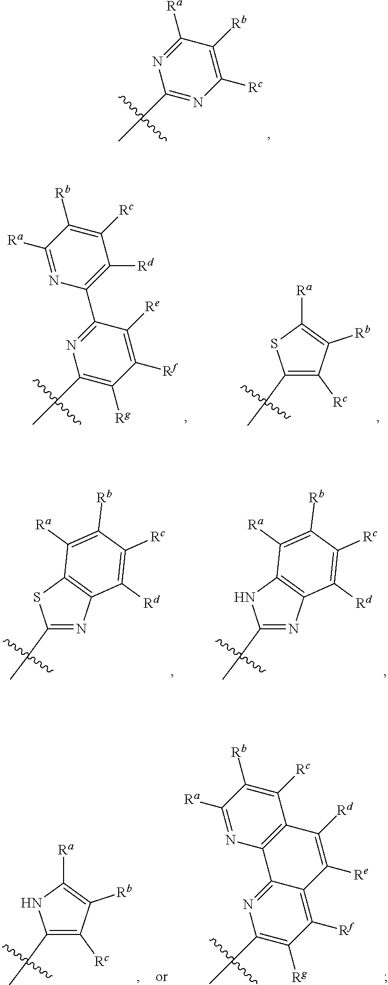
wherein:
R^{a} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{b} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{c} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{d} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{e} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{f} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃,
R^{g} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃; and
R² at each occurrence is selected from the group consisting of: —CH₃, H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃.

15. The compound of claim 14, in which R² at each occurrence is —CH₃.

16. The compound of claim 14, in which R¹ at each occurrence is —CH₂OH, —C(═O)OH, —C(═O)NH₂, —CH₂NH₂, —CH₂NHCH₃, —CH₂N(CH₃)₂, meta-C₆H₄NH₂, meta-C₆H₄NHCH₃, meta-C₆H₄N(CH₃)₂, or para-C₆H₄N(CH₃)₂.

17. The compound of claim 14, in which the compound is selected from the group consisting of:

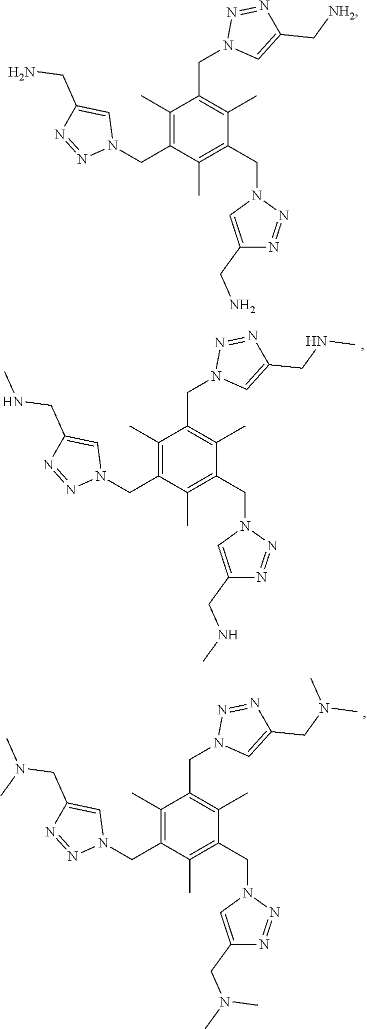

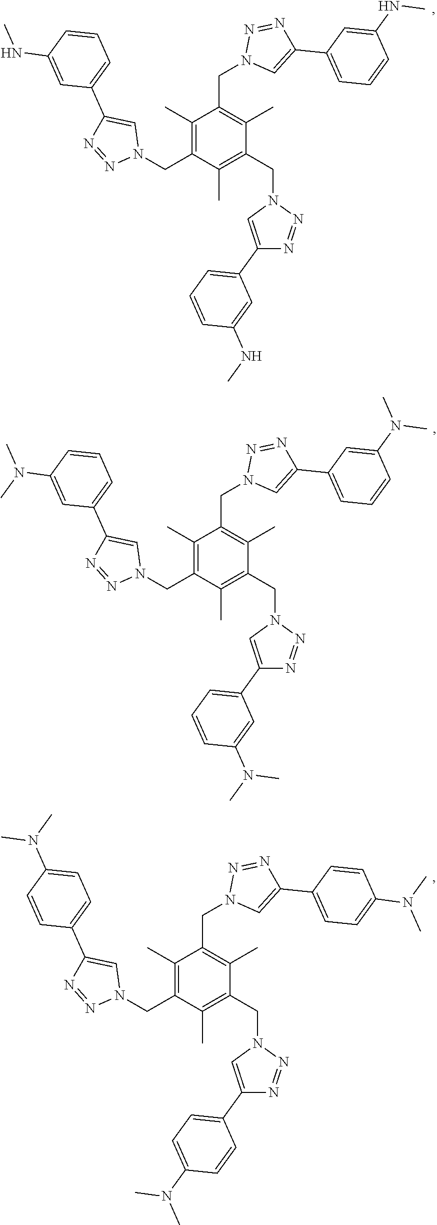

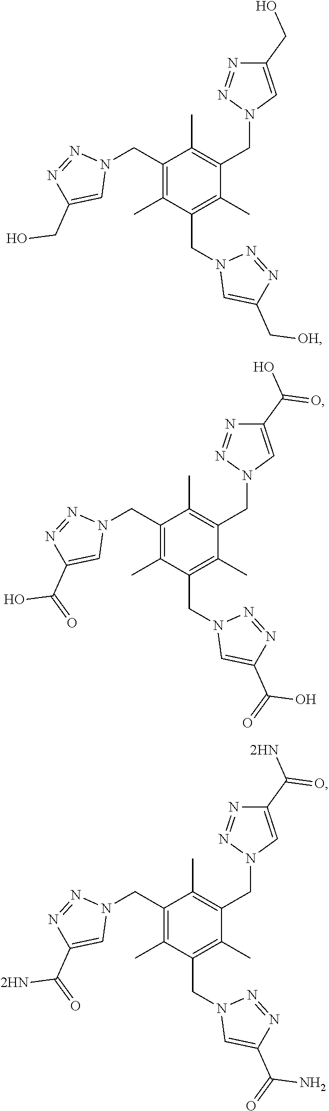

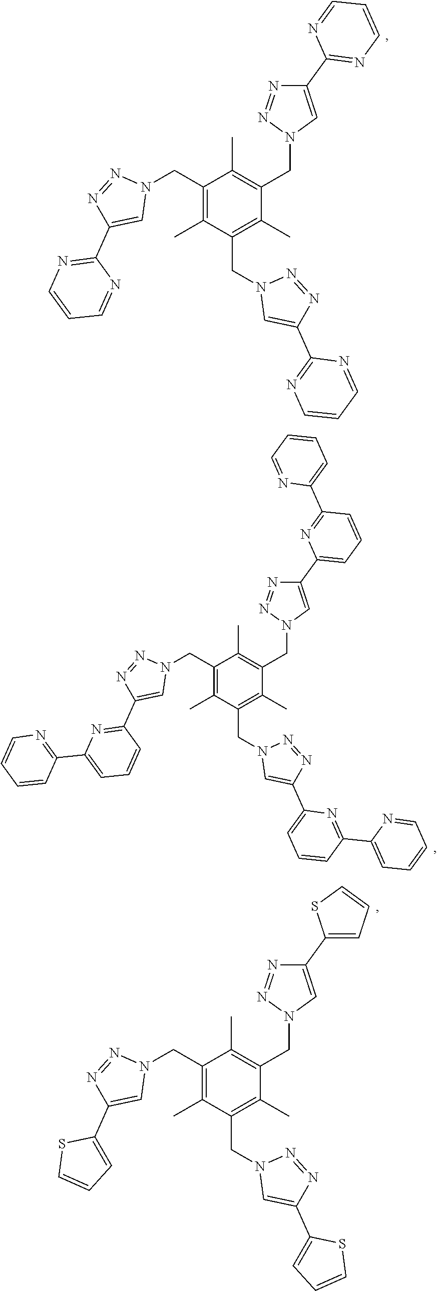

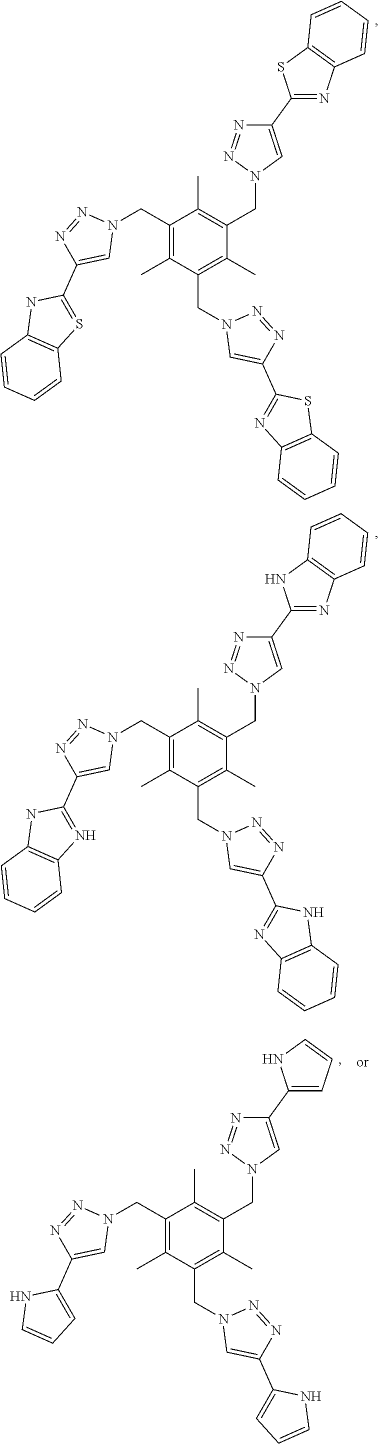

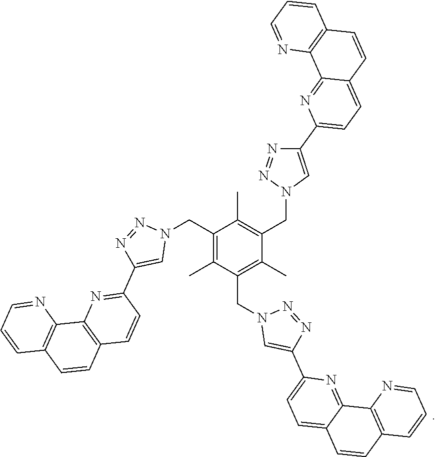 [SMILES: Cc1c(Cn2cc(-c3ccc4ccc5cccnc5c4n3)nn2)c(C)c(Cn2cc(-c3ccc4ccc5cccnc5c4n3)nn2)c(C)c1Cn1cc(-c2ccc3ccc4cccnc4c3n2)nn1]

18. A method of forming a compound of Formula (I): 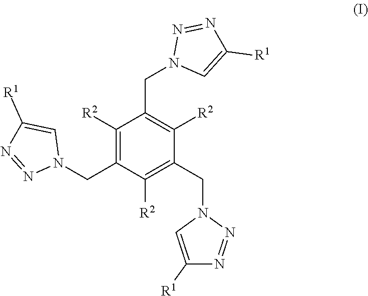
or a pharmaceutically acceptable salt thereof, wherein the method comprises the steps of:
reacting a compound of Formula (III): 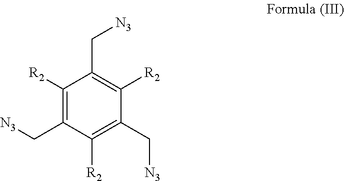
with a compound of Formula (IV) 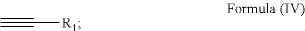
wherein:
R¹ is —CH₂OH, —C(═O)OH, —C(═O)NH₂, —CH₂NH₂, —CH₂NHCH₃, —CH₂N(CH₃)₂, meta-C₆H₄NH₂, meta-C₆H₄NHCH₃, meta-C₆H₄N(CH₃)₂,para-C₆H₄N(CH₃)₂, 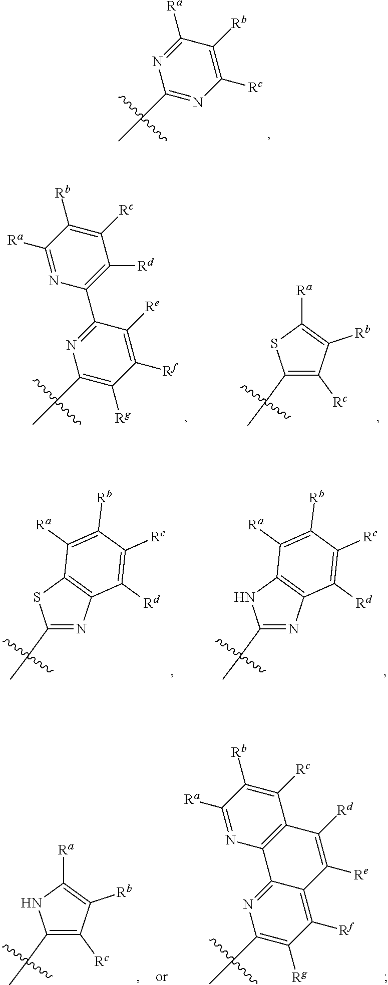
wherein:
R^{a} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{b} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{c} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{d} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{e} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{f} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃;
R^{g} at each occurrence is selected from the group consisting of: H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃; and
R² at each occurrence is selected from the group consisting of: —CH₃, H, unsubstituted C₁-C₁₀ alkyl, substituted C₁-C₁₀ alkyl, unsubstituted C₂-C₁₀ alkenyl, substituted C₂-C₁₀ alkenyl, unsubstituted C₂-C₁₀ alkynyl, substituted C₂-C₁₀ alkynyl, carboxylic acid, substituted thiol, unsubstituted thiol, substituted amine, unsubstituted amine, maleimide, F, Cl, Br, I, —NH₂, —OH or —N₃.

19. A method of claim 18, wherein R² at each occurrence is —CH₃.

20. A method of claim 18, wherein the compound of Formula (IV) comprises protected substituents; optionally, wherein the compound of Formula (IV) comprises Boc protected substituents.
